(19) United States
(12) Patent Application Publication
(10) Pub. No.: US 2024/0294584 A1
(43) Pub. Date: Sep. 5, 2024

(54) ELASTOMERIC PROTEINS

(71) Applicant: BOLT THREADS, INC., Emeryville, CA (US)

(72) Inventors: Joshua Kittleson, Berkeley, CA (US); Michael Lee, Berkeley, CA (US); David N. Breslauer, San Francisco, CA (US); Daniel M. Widmaier, San Francisco, CA (US)

(21) Appl. No.: 18/533,639

(22) Filed: Dec. 8, 2023

Related U.S. Application Data
(63) Continuation of application No. 17/219,277, filed on Mar. 31, 2021, now Pat. No. 11,858,971.
Application No. 17/219,277 is a continuation of application No. 16/477,133, filed on Jul. 10, 2019, now Pat. No. 10,988,515.
(60) Provisional application No. 62/446,230, filed on Jan. 13, 2017.

(51) Int. Cl. C07K 14/435; C12P 21/02

(52) U.S. Cl. CPC C07K 14/43563; C07K 14/43577; C07K 14/43581; C07K 14/4359; C12P 21/02

(57) ABSTRACT

This invention relates to elastomeric protein and elastomeric protein production. In particular, the invention is directed to elastomeric protein sequences, including methods and compositions for production of elastomeric protein sequences, such as expression constructs, and host cells, and including compositions generated from the elastomeric protein sequences.

### CROSS REFERENCE TO RELATED APPLICATIONS

This application is a continuation of U.S. application Ser. No. 17/219,277, filed Mar. 31, 2021, which is a continuation of U.S. application Ser. No. 16/477,133 (now U.S. Pat. No. 10,988,515), filed Jul. 10, 2019, which is a 371 national phase application of International Application No. PCT/US18/13839, filed Jan. 16, 2018, which claims the benefit of U.S. Provisional Application No. 62/446,230, filed Jan. 13, 2017, the entire disclosure of which is hereby incorporated by reference, in its entirety, for all purposes.

### INCORPORATION BY REFERENCE OF MATERIAL SUBMITTED ELECTRONICALLY

The application is being filed with a sequence listing in electronic format. The sequence listing is provided as a file entitled “50007B_Seqlisting.xml;” size: 85,506 bytes; created: Dec. 26, 2023. The information in the sequence listing is incorporated herein by reference in its entirety.

### TECHNICAL FIELD

The present disclosure relates generally to elastomeric protein and elastomeric protein production. Specifically, the present disclosure relates to elastomeric protein sequences, expression constructs, host cells, and solids.

### BACKGROUND

Elastomeric proteins are polypeptides that exhibit viscoelastic mechanical properties, and include elastin, resilin, abductin, and octopus arterial elastomers. Resilin is a particularly interesting elastomeric protein because it dissipates very little energy during loading and unloading. Resilin is found in many insects, and the low energy dissipation enables the extraordinary ability of many insect species to jump or pivot their wings very efficiently. The unique properties of resilin make it an interesting elastomeric material that could have many industrial applications. However, resilin exists in only very small quantities in nature, and therefore cannot be cost-effectively farmed by raising insects.

Variations of natural resilins and resilin-like proteins (based on resilin sequences) have been recombinantly produced by a number of groups in E. coli cultures, and have been isolated by lysing the cells to extract recombinantly expressed proteins, and using affinity chromatography techniques to purify (Elvin et al., 2005; Charati et al.; 2009, McGann et al., 2013). The recombinantly produced resilin and resilin-like proteins have been cross-linked targeting the tyrosine residues that also form the cross-links in natural resilin (see, for example, Elvin et al., 2005; Qin et al., 2011). Recombinantly produced resilin has also been cross-linked targeting lysine residues (Li et al., 2011) or cysteine residues (McGann et al., 2013). Cross-linked recombinantly produced resilin and resilin-like proteins have shown mechanical properties similar to those of natural resilin, with resilience values in excess of 90% (Elvin et al., 2005, Qin et al., 2011, Li et al., 2011).

In one study, 70-80 mg of recombinant resilin-like protein were produced per liter of E. coli culture, and the resilin-like protein was purified by Ni-NTA affinity chromatography (Charati et al., 2009). More efficient expression systems have been developed, which have produced 300-450 mg/L of recombinant resilin-like proteins from E. coli host cells (Lyons, et al., 2009). More efficient methods have also been developed for purifying the resilin-like proteins from lysed E. coli host cells, based on salt precipitation followed by heating (Qin et al., 2011; Lyons et al., 2009). However, improved systems for expressing and purifying elastomeric proteins (e.g., resilin and resilin-like proteins) with greater productivity are desired to provide more efficient protein production at larger scales.

At least one drawback of recovering expressed proteins by lysing cells followed by simple precipitation-based purification techniques is that the resulting proteins tend to have low purity due to cellular proteins from the lysed cells contaminating the target protein. Low purity can result in a variety of product defects, including low resilience. Furthermore, intracellular accumulation of protein can lead to toxicity and therefore decreased efficiency of production of recombinant elastomeric proteins. What is needed, therefore, are improved methods for expression and purification of recombinant elastomeric proteins that include methods to recover elastomeric proteins from extracellular portions. What is also needed are improved methods for expression and purification of recombinant elastomeric proteins (e.g., resilin and resilin-like proteins) that have a greater production efficiency.

### SUMMARY OF THE INVENTION

According to some embodiments, provided herein is a method for producing a composition comprising a recombinant resilin protein, the method comprising: culturing a population of recombinant host cells in a fermentation, wherein said recombinant host cells comprise a vector comprising a secreted resilin coding sequence, and wherein said recombinant host cells secrete a recombinant resilin protein encoded by said secreted resilin coding sequence; and purifying said recombinant resilin protein from said fermentation.

In some embodiments, the recombinant resilin protein is a full-length or truncated native resilin. In some embodiments, the native resilin is from an organism selected from the group consisting of: Drosophila sechellia, Acromyrmex echinatior, Aeshna, Haematobia irritans, Ctenocephalides felis, Bombus terrestris, Tribolium castaneum, Apis mellifera, Nasonia vitripennis, Pediculus humanus corporis, Anopheles gambiae, Glossina morsitans, Atta cephalotes, Anopheles darlingi, Acyrthosiphon pisum, Drosophila virilis, Drosophila erecta, Lutzomyia longipalpis, Rhodnius prolixus, Solenopsis invicta, Culex quinquefasciatus, Bactrocera cucurbitae, and Trichogramma pretiosum. In some embodiments, the recombinant resilin protein comprises SEQ ID NO: 1. In some embodiments, the recombinant resilin protein comprises SEQ ID NO: 4.

In some embodiments, the recombinant resilin protein comprises an alpha mating factor secretion signal. In some embodiments, the recombinant resilin protein comprises a FLAG-tag. In some embodiments, the vector comprises more than one secreted resilin coding sequence.

In some embodiments, the recombinant host cells are yeast cells. In some embodiments, the yeast cells are methylotrophic yeast cells. In some embodiments, the recombinant host cells are a species selected from the group consisting of: Pichia (Komagataella) pastoris, Hansenula polymorpha, Arxula adeninivorans, Yarrowia lipolytica, Pichia (Scheffersomyces) stipitis, Pichia methanolica, Saccharomyces cerevisiae, and Kluyveromyces lactis.

In some embodiments, the recombinant host cells produce the recombinant resilin at a rate of greater than 2 mg resilin/g dry cell weight/hour. In some embodiments, the recombinant host cells produce a secreted fraction of the recombinant resilin that is greater than 50% as compared to the total recombinant resilin protein expressed by the recombinant host cells. In some embodiments, the recombinant host cells secrete the recombinant resilin at a rate of greater than 2 mg resilin/g dry cell weight/hour. In some embodiments, greater than 80% of the recombinant resilin is outside of the recombinant host cells in said fermentation. In some embodiments, the fermentation comprises at least 2 g recombinant resilin/L.

In some embodiments, purifying said recombinant resilin protein comprises: generating a first pellet fraction and a first supernatant fraction by centrifuging the fermentation; and isolating the recombinant resilin protein from the first pellet fraction. In some embodiments, purifying said recombinant resilin protein further comprises: adding a chaotrope to the first pellet fraction to generate a solution in which the recombinant resilin protein is soluble; generating a second supernatant fraction and a second pellet fraction by centrifuging the first pellet fraction comprising said chaotrope; and isolating the soluble full-length resilin from the second supernatant fraction.

In some embodiments, provided herein is a vector comprising a secreted resilin coding sequence. In some embodiments, the secreted resilin coding sequence encodes a full-length or truncated native resilin. In some embodiments, the secreted resilin coding sequence encodes a modified full-length or truncated native resilin. In some embodiments, the modified resilin comprises an addition, subtraction, replacement, or change in position of an amino acid residue capable of cross-linking to another resilin.

In some embodiments, the full-length or truncated native resilin is from an organism selected from the group consisting of: Drosophila sechellia, Acromyrmex echinatior, Aeshna, Haematobia irritans, Ctenocephalides felis, Bombus terrestris, Tribolium castaneum, Apis mellifera, Nasonia vitripennis, Pediculus humanus corporis, Anopheles gambiae, Glossina morsitans, Atta cephalotes, Anopheles darlingi, Acyrthosiphon pisum, Drosophila virilis, Drosophila erecta, Lutzomyia longipalpis, Rhodnius prolixus, Solenopsis invicta, Culex quinquefasciatus, Bactrocera cucurbitae, and Trichogramma pretiosum.

In some embodiments, the secreted resilin coding sequences encodes a polypeptide comprising SEQ ID NO: 1. In some embodiments, the secreted resilin coding sequence encodes a polypeptide comprising SEQ ID NO: 4. In some embodiments, the secreted resilin coding sequence encodes a recombinant resilin comprising one or more A-repeats or quasi-A-repeats. In some embodiments, the secreted resilin coding sequence encodes a recombinant resilin comprising one or more B-repeats or quasi-B-repeats. In some embodiments, the secreted resilin coding sequence encodes a recombinant resilin comprising either one or more A-repeats or quasi-A-repeats or one or more B-repeats or quasi-B repeats but not both. In some embodiments, the secreted resilin coding sequence encodes a recombinant resilin comprising one or more A-repeats or quasi-A-repeats and one or more B-repeats or quasi-B-repeats.

In some embodiments, the recombinant resilin further comprises a chitin binding domain. In some embodiments, the secreted resilin coding sequence encodes a polypeptide comprising an alpha mating factor secretion signal. In some embodiments, the secreted resilin coding sequence comprises a FLAG-tag.

In some embodiments, the vector comprises more than one secreted resilin coding sequence. In some embodiments, the vector comprises 3 secreted resilin coding sequences. In some embodiments, the secreted resilin coding sequence is operatively linked to a constitutive or inducible promoter.

Also provided herein, according to some embodiments, is a recombinant host cell comprising one or more vectors comprising a secreted resilin coding sequence. In some embodiments, the recombinant host cell is a yeast cell. In some embodiments, the yeast cell is a methylotrophic yeast cell. In some embodiments, the recombinant host cell is a species selected from the group consisting of: Pichia (Komagataella) pastoris, Hansenula polymorpha, Arxula adeninivorans, Yarrowia lipolytica, Pichia (Scheffersomyces) stipitis, Pichia methanolica, Saccharomyces cerevisiae, and Kluyveromyces lactis.

In some embodiments, the recombinant host cell comprises 3 vectors comprising a secreted resilin coding sequence.

In some embodiments, the recombinant host cell produces recombinant resilin at a rate of greater than 2 mg resilin/g dry cell weight/hour. In some embodiments, the recombinant host cell has a secreted fraction of recombinant resilin that is greater than 50%. In some embodiments, the recombinant host cell secretes resilin at a rate of greater than 2 mg resilin/g dry cell weight/hour.

Also provided herein, according to some embodiments, is a fermentation comprising a recombinant host cell comprising one or more vectors comprising a secreted resilin coding sequence and a culture medium suitable for growing the recombinant host cell.

In some embodiments, the fermentation comprises at least 2 g recombinant resilin/L.

In some embodiments of the fermentation, greater than 80% of recombinant resilin is outside of the recombinant host cells.

In some embodiments of the fermentation, the recombinant resilin is full-length recombinant resilin.

Also provided herein, according to some embodiments, is a composition comprising recombinant resilin derived from a fermentation comprising a recombinant host cell comprising one or more vectors comprising a secreted resilin coding sequence and a culture medium suitable for growing the recombinant host cell. In some embodiments, the composition comprises at least 60% by weight of recombinant resilin.

In some embodiments, the composition has similar properties compared to compositions comprising similar amounts of native resilins. In some embodiments, the composition has different properties compared to compositions comprising similar amounts of native resilins.

In some embodiments, the composition comprises a resilience of greater than 50%. In some embodiments, the composition comprises has a compressive elastic modulus of less than 10 MPa. In some embodiments, the composition has a tensile elastic modulus of less than 10 MPa. In some embodiments, the composition has a shear modulus of less than 1 MPa. In some embodiments, the composition has an extension to break of greater than 1%. In some embodiments, the composition has a maximum tensile strength of greater than 0.1 kPa. In some embodiments, the composition has a Shore 00 Hardness of less than 90. In some embodiments, the composition comprises full-length resilin.

Also provided herein, according to some embodiments, is a method for producing a composition comprising recombinant resilin, the method comprising the step of culturing a recombinant host cell comprising one or more vectors comprising a secreted resilin coding sequence to produce a fermentation under conditions that promote secretion of recombinant resilin from the recombinant host cell.

In some embodiments, the method for producing a composition comprising recombinant resilin further comprises the step of purifying said recombinant resilin to produce full-length native resilin. In some embodiments, purifying said recombinant resilin to produce full-length native resilin comprises: generating a first pellet fraction and a first supernatant fraction by centrifuging the fermentation; and isolating the recombinant resilin protein from the first pellet fraction. In some embodiments, isolating the recombinant resilin protein from the first pellet fraction comprises: adding a chaotrope to the first pellet fraction to generate a solution in which the recombinant resilin protein is soluble; generating a second supernatant fraction and a second pellet fraction by centrifuging the first pellet fraction comprising said chaotrope; and isolating the recombinant resilin protein from the second supernatant fraction.

In some embodiments, the method for producing a composition comprising recombinant resilin further comprises the step of cross-linking a plurality of said recombinant resilins. In some embodiments, said cross-linking is enzymatic cross-linking. In some embodiments, said cross-linking is photochemical cross-linking. In some embodiments, the recombinant resilin protein comprises a full-length resilin protein.

Also provided herein, according to some embodiments, is a fermentation comprising a culture medium and a recombinant host cell, wherein the recombinant host cell comprises a vector, wherein the vector comprises a secreted resilin coding sequence, and wherein the recombinant host cell secretes recombinant resilin at a rate of at least 2 mg/g dry cell weight/hour.

### BRIEF DESCRIPTION OF THE DRAWINGS

FIG. 1 schematically illustrates the structure of an exemplary resilin.

FIG. 2 is a flow diagram of methods for the production of compositions comprising recombinant resilins.

FIG. 3 is an illustrative map of a vector that comprises 3 secreted resilin coding sequences.

FIG. 4A shows expression and secretion of 3X FLAG-tagged recombinant resilins in Pichia pastoris (Komagataella phaffii) recombinant host cells as assayed by ELISA. FIG. 4B shows expression of recombinant resilins in Pichia pastoris (Komagataella phaffii) recombinant host cells as assayed by Western blot (top; 3xFLAG-tagged recombinant resilins), and Coomassie (bottom; untagged recombinant resilins).

FIG. 5A shows productivities of recombinant host cells producing recombinant resilins in rich media. FIG. 5B shows productivities of recombinant host cells producing recombinant resilins in minimal media.

FIG. 6 shows purification of 2 secreted recombinant resilins from 500 mL BMGY flask growth. For each sample, Lane 1 is the original supernatant; Lane 2 is the supernatant after precipitation; Lane 3 is the dialyzed precipitate; Lane 4 is the heat-denatured proteins; and Lane 5 is the final purified recombinant resilin.

FIG. 7 shows photographs of proteinaceous block co-polymers comprising cross-linked purified recombinant resilins in various shapes and forms.

FIG. 8 shows photographs of the compression of a proteinaceous block co-polymer comprising cross-linked recombinant resilin.

FIG. 9 is an image of a gel showing the resulting bands from recombinant resilin compositions purified by selected methods described herein.

FIG. 10 is the full-length Drosophila sechellia resilin sequence (Ds_ACB) (SEQ ID NO: 10) that is expressed along with signal sequences that are later cleaved, according to some embodiments of the invention.

The figures depict various embodiments of the present disclosure for purposes of illustration only. One skilled in the art will readily recognize from the following discussion that alternative embodiments of the structures and methods illustrated herein may be employed without departing from the principles described herein.

### Definitions

Unless defined otherwise, all technical and scientific terms used herein have the same meaning as is commonly understood by one of ordinary skill in the art to which this disclosure pertains.

The terms “a” and “an” and “the” and similar referents as used herein refer to both the singular and the plural, unless otherwise indicated herein or clearly contradicted by context.

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

Amino acids can be referred to by their single-letter codes or by their three-letter codes. The single-letter codes, amino acid names, and three-letter codes are as follows: G-Glycine (Gly), P-Proline (Pro), A-Alanine (Ala), V-Valine (Val), L-Leucine (Leu), I-Isoleucine (Ile), M-Methionine (Met), C-Cysteine (Cys), F-Phenylalanine (Phe), Y-Tyrosine (Tyr), W-Tryptophan (Trp), H-Histidine (His), K-Lysine (Lys), R-Arginine (Arg), Q-Glutamine (Gln), N-Asparagine (Asn), E-Glutamic Acid (Glu), D-Aspartic Acid (Asp), S-Serine (Ser), T-Threonine (Thr).

The terms “including,” “includes,” “having,” “has,” “with,” or variants thereof are intended to be inclusive in a manner similar to the term “comprising”.

The term “microbe” as used herein refers to a microorganism, and refers to a unicellular organism. As used herein, the term includes all bacteria, all archaea, unicellular protista, unicellular animals, unicellular plants, unicellular fungi, unicellular algae, all protozoa, and all chromista.

The term “native” as used herein refers to compositions found in nature in their natural, unmodified state.

The terms “optional” or “optionally” mean that the feature or structure may or may not be present, or that an event or circumstance may or may not occur, and that the description includes instances where a particular feature or structure is present and instances where the feature or structure is absent, or instances where the event or circumstance occurs and instances where the event or circumstance does not occur.

The term “secreted fraction” as used herein refers to the fraction of recombinant resilins that are secreted from cells compared to the total resilins produced by the cells.

The term “secretion signal” as used herein refers to a short peptide that when fused to a polypeptide mediates the secretion of that polypeptide from a cell.

The term “secreted resilin coding sequence” as used herein refers to a nucleotide sequence that encodes a resilin as provided herein fused at its N-terminus to a secretion signal and optionally at its C-terminus to a tag peptide or polypeptide.

The term “recombinant” as used herein in reference to a polypeptide (e.g., resilin) refers to a polypeptide that is produced in a recombinant host cell, or to a polypeptide that is synthesized from a recombinant nucleic acid.

The term “recombinant host cell” as used herein refers to a host cell that comprises a recombinant nucleic acid.

The term “recombinant nucleic acid” as used herein refers to a nucleic acid that is removed from its naturally occurring environment, or a nucleic acid that is not associated with all or a portion of a nucleic acid abutting or proximal to the nucleic acid when it is found in nature, or a nucleic acid that is operatively linked to a nucleic acid that it is not linked to in nature, or a nucleic acid that does not occur in nature, or a nucleic acid that contains a modification that is not found in that nucleic acid in nature (e.g., insertion, deletion, or point mutation introduced artificially, e.g., by human intervention), or a nucleic acid that is integrated into a chromosome at a heterologous site. The term includes cloned DNA isolates and nucleic acids that comprise chemically-synthesized nucleotide analog.

The term “vector” as used herein refers to a nucleic acid molecule capable of transporting another nucleic acid to which it has been linked. One type of vector is a “plasmid,” which generally refers to a circular double stranded DNA loop into which additional DNA segments can be ligated, but also includes linear double-stranded molecules such as those resulting from amplification by the polymerase chain reaction (PCR) or from treatment of a circular plasmid with a restriction enzyme. Other vectors include bacteriophages, cosmids, bacterial artificial chromosomes (BAC), and yeast artificial chromosomes (YAC). Another type of vector is a viral vector, wherein additional DNA segments can be ligated into the viral genome. Certain vectors are capable of autonomous replication in a cell into which they are introduced (e.g., vectors having an origin of replication that functions in the cell). Other vectors can be integrated into the genome of a cell upon introduction into the cell, and are thereby replicated along with the cell genome.

The term “repeat” as used herein, in reference to an amino acid or nucleic acid sequence, refers to a sub-sequence that is present more than once in a polynucleotide or polypeptide (e.g., a concatenated sequence). A polynucleotide or polypeptide can have a direct repetition of the repeat sequence without any intervening sequence, or can have non-consecutive repetition of the repeat sequence with intervening sequences. The term “quasi-repeat” as used herein, in reference to amino acid or nucleic acid sequences, is a sub-sequence that is inexactly repeated (i.e., wherein some portion of the quasi-repeat subsequence is variable between quasi-repeats) across a polynucleotide or polypeptide. Repeating polypeptides and DNA molecules (or portions of polypeptides or DNA molecules) can be made up of either repeat sub-sequences (i.e., exact repeats) or quasi-repeat sub-sequences (i.e., inexact repeats).

The term “native resilin” as used herein refers to an elastomeric polypeptide or protein produced by insects. GenBank Accession Nos. of non-limiting examples of native resilin includes the following NCBI sequence numbers: NP 995860 (Drosophila melanogaster), NP 611157 (Drosophila melanogaster), Q9V7U0 (Drosophila melanogaster), AAS64829, AAF57953 (Drosophila melanogaster), XP 001817028 (Tribolium castaneum) and XP001947408 (Acyrthosiphon pisum).

The term “modified” as used herein refers to a protein or polypeptide sequence that differs in composition from a native protein or polypeptide sequence, where the functional properties are preserved to within 10% of the native protein or polypeptide properties. In some embodiments, the difference between the modified protein or polypeptide and the native protein or polypeptide can be in primary sequence (e.g., one or more amino acids are removed, inserted or substituted) or post-translation modifications (e.g., glycosylation, phosphorylation). Amino acid deletion refers to removal of one or more amino acids from a protein. Amino acid insertion refers to one or more amino acid residues being introduced in a protein or polypeptide. Amino acid insertions may comprise N-terminal and/or C-terminal fusions as well as intra-sequence insertions of single or multiple amino acids. Amino acid substitution includes non-conservative or conservative substitution, where conservative amino acid substitution tables are well known in the art (see for example Creighton (1984) Proteins. W. H. Freeman and Company (Eds)). In some embodiments, the modified protein or polypeptide and the native protein or polypeptide amino acid or nucleotide sequence identity is at least 60%, at least 65%, at least 70%, at least 75%, at least 80%, at least 85%, at least 90%, at least 95%, or at least 98% of the amino acids or nucleotide bases.

The term “truncated” as used herein refers to a protein or polypeptide sequence that is shorter in length than a native protein or polypeptide. In some embodiments, the truncated protein or polypeptide can be greater than 10%, or greater than 20%, or greater than 30%, or greater than 40%, or greater than 50%, or greater than 60%, or greater than 70%, or greater than 80%, or greater than 90% of the length of the native protein or polypeptide.

The term “homolog” or “substantial similarity,” as used herein, when referring to a polypeptide, nucleic acid or fragment thereof, indicates that, when optimally aligned with appropriate amino acid or nucleotide insertions or deletions with another amino acid or nucleic acid (or its complementary strand), there is amino acid or nucleotide sequence identity in at least 60%, at least 65%, at least 70%, at least 75%, at least 80%, at least 85%, at least 90%, at least 95%, or at least 98% of the amino acids or nucleotide bases, as measured by any well-known algorithm of sequence identity, such as FASTA, BLAST or Gap, as discussed above.

The term “resilin” as used herein refers to a protein or a polypeptide, capable of cross-linking to form an elastomer, where the protein or polypeptide is a native resilin, or a native resilin that is modified, or a native resilin that is truncated. Resilins of the present invention are preferably recombinant resilins. In some embodiments, recombinant resilins comprise a natural or modified (e.g., truncated or concatenated) nucleotide sequence coding for resilin or resilin fragments (e.g., isolated from insects), heterologously expressed and secreted from a host cell. In preferred embodiments, the secreted recombinant resilin protein is collected from a solution extracellular to the host cell.

As used herein, the term “elastomer” refers to a polymer with viscoelasticity and typically weak inter-molecular forces (except for covalent cross-links between molecules, if they are present). Viscoelasticity is a property of materials that exhibit both viscous and elastic characteristics when undergoing deformation, and therefore exhibit time-dependent strain. Elasticity is associated with bond stretching along crystallographic planes in an ordered solid, and viscosity is the result of the diffusion of atoms or molecules inside an amorphous material. Elastomers that are viscoelastic, therefore, generally have low Young's modulus and high failure strain compared with other materials. Due to the viscous component of the material, viscoelastic materials dissipate energy when a load is applied and then removed. This phenomenon is observed as hysteresis in the stress-strain curve of viscoelastic materials. As a load is applied there is a particular stress-strain curve, and as the load is removed the stress-strain curve upon unloading is different than that of the curve during loading. The energy dissipated is the area between the loading and unloading curves.

Recitation of ranges of values herein are merely intended to serve as a shorthand method of referring individually to each separate value inclusively falling within the range, unless otherwise indicated herein, and each separate value is incorporated into the specification as if it were individually recited herein.

### DETAILED DESCRIPTION

Provided herein are compositions comprising recombinant resilins, and methods for their production.

Resilins have many unique properties compared to petroleum-based elastomers. Most notably, resilin has an extreme elastic efficiency, where very little of the energy input into deformation is lost as heat. Other desirable properties of resilin include, for example, desirable resilience, compressive elastic modulus, tensile elastic modulus, shear modulus, extension to break, maximum tensile strength, hardness, rebound, and compression set. Moreover, resilin is a protein, and therefore can be biodegraded, which makes it more environmentally friendly than petroleum-based polymers. Also, resilin is biocompatible and can therefore be used in applications that involve contact with humans or animals. Lastly, the mechanical properties of recombinant resilins can be tuned through varying protein sequence, protein structure, amount of intermolecular cross-linking and processing variables to produce elastomers designed for a universe of specific applications.

In some embodiments, the methods and compositions provided herein provide efficient means for producing large quantities of recombinant resilins. In some embodiments, large quantities of resilin and resilin-like polypeptides are obtained using recombinant host cells that secrete recombinant resilins via their secretory pathways. Such secretion of recombinant resilins a) avoids toxicity from intracellular accumulation of recombinant resilins, b) simplifies purification by eliminating cell disruption or protein refolding processes, and c) provides opportunities for post-translational events (e.g., proteolytic maturation, glycosylation, disulfide bond formation) that can modulate the properties of the recombinant resilins.

### COMPOSITIONS COMPRISING RECOMBINANT RESILINS

In some embodiments, the compositions provided herein comprise recombinant resilins.

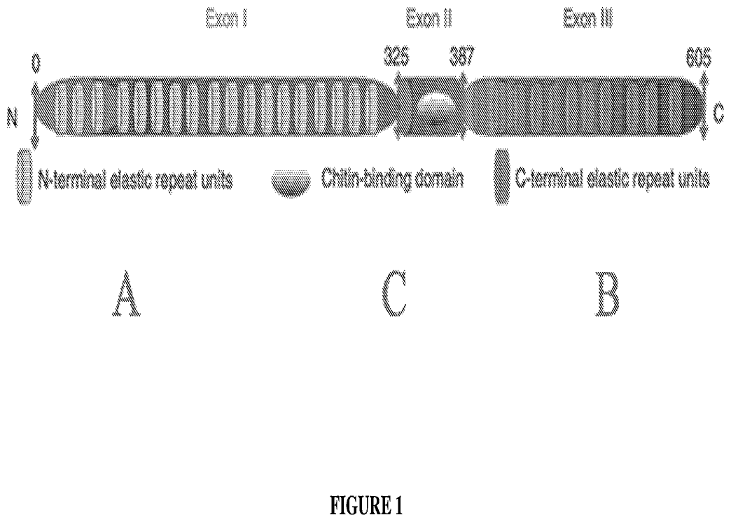

FIG. 1 illustrates an example of a native resilin, which contains an N-terminal A-domain comprising a plurality of repeat units comprising the consensus amino acid sequence YGXP (“A-repeat”), where X is any amino acid; a chitin-binding type RR-2 (C) domain (Pfam reference PF00379; Rebers JE & Willis, JH. A conserved domain in anthropod cuticular proteins binds chitin. Insect Biochem Mol Biol 31:1083-1093); and a C-terminal B-domain comprising a plurality of repeat units comprising the consensus amino acid sequence UYZXZ (“B-repeat”), where U is glycine or serine; Z is serine, glycine, arginine, or proline; and X is any amino acid. Not all naturally occurring resilins have A-, C-, and B-domains. Native resilins produced by various insects typically have inexact repeats (i.e., quasi-repeats) within the A- and/or B-domains with some amino acid variation between the quasi-repeats.

In some embodiments, the recombinant resilins provided herein comprise one or more A-repeats. In some embodiments, the recombinant resilins comprise N-terminal A-domains comprising a plurality of blocks of A-repeat and/or quasi-A-repeat amino acid sub-sequences each with the consensus sequence SXXYGXP, where S is serine, X is an amino acid, Y is tyrosine, G is glycine, and P is proline.

In some embodiments, the recombinant resilins provided herein comprise one or more B-repeats. In some embodiments, the recombinant resilins comprise a C-terminal B-domain comprising a plurality of blocks of B-repeat and/or quasi-B-repeat amino acid sub-sequences each with the consensus sequence GYZXZZX and/or SYZXZZX, where G is glycine; Y is tyrosine; Z is serine, glycine, proline, or arginine; S is serine; and X is any amino acid.

In some embodiments, the recombinant resilins provided herein comprise one or more A-repeats. In some such embodiments, the recombinant resilins comprise between 1 and 100 A-repeats, or from 2 to 50 A-repeats, or from 5 to 50 A-repeats, or from 5 to 20 A-repeats.

In some embodiments, the recombinant resilins comprise one or more consensus sequences described by the formula,

(X₁-X₂-X₃-X₄)ₙ (1)
wherein the brackets delineate a repeat or quasi-repeat of the consensus sequence;
wherein n describes the number of A-repeats or quasi-A-repeats, and is from 1 to 100, or from 2 to 50, or from 5 to 50, or from 5 to 20;
wherein X₁ is a motif that is 4 amino acids in length, wherein the first amino acid of X₁ is Y, and wherein the remaining amino acids of X₁ are GAP, GLP, GPP, GTP, or GVP;
wherein X₂ is a motif that is from 3 to 20 amino acids in length;
wherein X₂ comprises GGG, GGGG, N, NG, NN, NGN, NGNG, GQGG, GQGN, GQGQ,
GQGQG, or 3 or more glycine residues, or wherein 50% or more of the residues of X₂ are either glycine or asparagine, or wherein 60% or more of the residues of X₂ are either glycine or asparagine, or wherein 70% or more of the residues of X₂ are either glycine or asparagine, or wherein 80% or more of the residues of X₂ are either glycine or asparagine;
wherein X₃ is a motif that is from 2 to 6 amino acids in length, wherein X₃ is GG, LS, APS, GAG, GGG, KPS, RPS, or GGGG; and
wherein X₄ is a motif that is from 1 to 2 amino acids in length, wherein X₄ is S, D, T, N, L, DS, DT, LS, SS, ST, TN, or TS.

In some such embodiments, the recombinant resilins comprise motifs X₁, X₂, X₃, and X₄ whereas in other embodiments, the recombinant resilins comprise motifs X₁, X₂, X₃, or X₄, or combinations thereof.

In some embodiments, the recombinant resilins provided herein comprise one or more B-repeats. In some such embodiments, the recombinant resilins comprise between 1 and 100 B-repeats, or from 2 to 50 A-repeats, or from 5 to 50 A-repeats, or from 5 to 20 A-repeats.

In some embodiments, the recombinant resilins comprise one or more consensus sequences described by the formula,

(X₁₁-X₁₂-X₁₃)ₘ (2)
wherein the brackets delineate a repeat or quasi-repeat of the consensus sequence;
wherein m describes the number of B-repeats or quasi-B-repeats, and is from 1 to 100;
wherein X₁₁ is a motif that is from 1 to 5 amino acids in length, the first amino acid is Y, and where the remaining amino acids can comprise GAP, GPP, SSG, or SGG;
wherein X₁₂ is a motif that is from 2 to 5 amino acids in length and comprises GQ, GN, RPG, RPGGQ, RPGGN, SSS, SKG, or SN; and wherein X₁₃ is a motif that is from 4 to 30 amino acids in length and comprises GG, DLG, GFG, GGG, RDG, SGG, SSS, GGSF, GNGG, GGAGG, or 3 or more glycine residues, or 30% or more of the residues are glycine, or 40% or more of the residues are glycine, or 50% or more of the residues are glycine, or 60% or more of the residues are glycine.

In some such embodiments, the recombinant resilins comprise motifs X₁₁, X₁₂, and X₁₃ whereas in other such embodiments, the recombinant resilins comprise motifs X₁₁, X₁₂, or X₁₃, or combinations thereof.

In some embodiments, the recombinant resilins provided herein comprise one or more A-repeats, one or more B-repeats, and/or one or more C-domain. In some embodiments, the recombinant resilins comprise one or more A-repeats or one or more B-repeats but not both. In some embodiments, the recombinant resilins comprise one or more A-repeats but not B-repeats or C-domains. In some embodiments, the recombinant resilins comprise one or more B-repeats but not A-repeats or C-domains. In embodiments in which the recombinant resilins comprise a C-domain, the C-domain can be situated either on the N-terminal or the C-terminal sides of the A-repeats or B-repeats, or between the A-repeats and the B-repeats.

In some embodiments, the recombinant resilins further comprise the sequence XXEPPVSYLPPS, where X is any amino acid. In some such embodiments, the sequence is located on the N-terminal side of an A-repeat or B-repeat.

In some embodiments, the recombinant resilins are full-length native resilins expressed in a non-native environment. In some embodiments, the recombinant resilins comprise a truncated version of native resilins. In some embodiments, the truncated native resilins comprise at least one A-repeat. In some embodiments, the truncated native resilins comprise at least one B-repeat. Non-limiting examples of full-length and truncated native resilins are provided as SEQ ID NOs: 1 through 44. In some embodiments, the recombinant resilins are full-length Drosophila sechellia resilin (SEQ ID NO: 1). In some embodiments, the recombinant resilins are truncated Acromyrmex echinatior resilin (SEQ ID NO: 4). In some embodiments, the recombinant resilins are full-length or truncated native resilins that are cross-linked in a non-native manner (e.g., less or more cross-linking, cross-linking via different amino acid residues).

In some embodiments, the recombinant resilins are modified full-length or truncated native resilins. In some embodiments, the recombinant resilins are at least 60%, at least 65%, at least 70%, at least 75%, at least 80%, at least 85%, at least 90%, at least 95%, or at least 98% identical to a full-length or truncated native resilin. In some embodiments, the recombinant resilins are at least 60%, at least 65%, at least 70%, at least 75%, at least 80%, at least 85%, at least 90%, at least 95%, or at least 98% identical to full-length Drosophila sechellia resilin (SEQ ID NO: 1). In some embodiments, the recombinant resilins are at least 60%, at least 65%, at least 70%, at least 75%, at least 80%, at least 85%, at least 90%, at least 95%, or at least 98% identical to truncated Acromyrmex echinatior resilin (SEQ ID NO: 4).

There are a number of different algorithms known in the art which can be used to measure nucleotide sequence or protein sequence identity. For instance, polynucleotide sequences can be compared using FASTA, Gap, or Bestfit, which are programs in Wisconsin Package Version 10.0, Genetics Computer Group (GCG), Madison, Wis. FASTA provides alignments and percent sequence identity of the regions of the best overlap between the query and search sequences. See, e.g., Pearson, Methods Enzymol. 183:63-98, 1990 (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410, 1990; Gish and States, Nature Genet. 3:266-272, 1993; Madden et al., Meth. Enzymol. 266:131-141, 1996; Altschul et al., Nucleic Acids Res. 25:3389-3402, 1997; Zhang and Madden, Genome Res. 7:649-656, 1997, especially blastp or tblastn (Altschul et al., Nucleic Acids Res. 25:3389-3402, 1997.

In some embodiments, the modified resilins differ from full-length or truncated native resilins in amino acid residues that are post-translationally modified (e.g., glycosylated, phosphorylated) such that the modified resilins have one or more different locations and/or different amounts and/or different types of post-translational modifications than the full-length or truncated native resilins. In some embodiments, the modified resilins differ from full-length or truncated native resilins in amino acid residues that are involved in cross-linking such that the modified resilins have one or more different locations and/or different amounts and/or different types of amino acids that are involved in cross-linking than full-length or truncated native resilins. In some such embodiments, the modified resilins differ from the full-length or truncated native resilin in comprising one or more additional or fewer tyrosine residues, one or more additional or fewer lysine residues, and/or one or more additional or fewer cysteine residues.

In some embodiments, the recombinant resilins comprise concatenated native or truncated native resilins or concatenated modified resilins. In some embodiments, the concatenated native or truncated native resilins or concatenated modified resilins comprise at least 2 A-repeats (e.g., 2, 3, 4, 5, 6, 7, 8, 9, 10, or more). In some embodiments, the concatenated truncated native resilins or concatenated modified resilins comprise at least 2 B-repeats (e.g., 2, 3, 4, 5, 6, 7, 8, 9, 10, or more).

The compositions provided herein comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by weight of recombinant resilins. The recombinant resilins can be identical recombinant resilins or mixtures of recombinant resilins having at least 2 different amino acid sequences.

In some embodiments, the compositions provided herein have similar properties compared to compositions comprising native resilins. In other embodiments, the compositions provided herein have different properties compared to compositions comprising native resilins. Non-limited examples of such properties include resilience, compressive elastic modulus, tensile elastic modulus, shear modulus, extension to break, maximum tensile strength, hardness, rebound, and compression set. Parameters that can be modified to obtain compositions with specific mechanical properties include, for example, the length and/or sequence of the recombinant resilins, the extent and/or type of post-translational modifications of the recombinant resilins, and/or the extent and/or type of cross-linking of the recombinant resilins.

In some embodiments, mechanical properties such as maximum tensile strength, compressive elastic modulus, tensile elastic modulus, shear modulus, extension to break and resilience can be measured using many different types of tensile and compression systems that conduct stress-strain measurements on elastomeric samples. The resulting stress-strain curves, including curves with hysteresis, can be measured in tension or compression. In some embodiments, tension and compression test systems can apply a strain to a sample and measure the resulting force using a load cell. In some embodiments, the mechanical properties can be measured at the macroscopic scale (e.g., using macroscopic compression testers), microscopic, or nanoscopic scale (e.g., using atomic-force microscopy (AFM) or nanoindentation measurements). In some embodiments, the compressive mechanical properties of elastomers can be measured according to the standard ASTM D575-91(2012) Standard Test Methods for Rubber Properties in Compression. Mechanical measurements of elastomers in tension can be performed using ASTM D412-15a Standard Test Methods for Vulcanized Rubber and Thermoplastic Elastomers-Tension. In some embodiments, tear strength of elastomers can be performed using ASTM D624-00 Standard Test Method for Tear Strength of Conventional Vulcanized Rubber and Thermoplastic Elastomers. In some embodiments, mechanical properties of slab, bonded, and molded elastomers can be performed using ASTM D3574-11 Standard Test Methods for Flexible Cellular Materials-Slab, Bonded, and Molded Urethane Foams. In some embodiments, the mechanical properties of elastomers can be measured using ASTM D5992-96(2011) Standard Guide for Dynamic Testing of Vulcanized Rubber and Rubber-Like Materials Using Vibratory Methods.

In some embodiments, the compositions provided herein have a resilience of greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%.

In some embodiments, the compositions provided herein have a compressive elastic modulus of less than 10 MPa, less than 7 MPa, less than 5 MPa, less than 2 MPa, less than 1 MPa, less than 0.5 MPa, or less than 0.1 MPa; from 0.01 MPa to 10 MPa, 7 MPa, 5 MPa, 2 MPa, 1 MPa, 0.5 MPa, or 0.1 MPa; from 0.1 MPa to 10 MPa, 7 MPa, 5 MPa, 2 MPa, 1 MPa, or 0.5 MPa; from 0.5 MPa to 10 MPa, 7 MPa, 5 MPa, 2 MPa, or 1 MPa; from 1 MPa to 10 MPa, 7 MPa, 5 MPa, or 2 MPa; from 2 MPa to 10 MPa, 7 MPa, or 5 MPa; from 5 MPa to 10 MPa, or 7 MPa; or from 7 MPa to 10 MPa. In some embodiments, the compressive elastic modulus of a composition can be measured as defined by the ASTM D575-91(2012) Standard Test Methods for Rubber Properties in Compression.

In some embodiments, the compositions provided herein have a tensile elastic modulus of less than 10 MPa, less than 7 MPa, less than 5 MPa, less than 2 MPa, less than 1 MPa, less than 0.5 MPa, or less than 0.1 MPa; from 0.01 MPa to 10 MPa, 7 MPa, 5 MPa, 2 MPa, 1 MPa, or 0.5 MPa; from 0.5 MPa to 10 MPa, 7 MPa, 5 MPa, 2 MPa, or 1 MPa; from 1 MPa to 10 MPa, 7 MPa, 5 MPa, or 2 MPa; from 2 MPa to 10 MPa, 7 MPa, or 5 MPa; from 5 MPa to 10 MPa, or 7 MPa; or from 7 MPa to 10 MPa.

In some embodiments, the compositions provided herein have a shear modulus of less than 1 MPa, less than 100 kPa, less than 50 kPa, less than 20 kPa, less than 10 kPa, or less than 1 kPa; from 0.1 kPa to 1 MPa, 100 kPa, 50 kPa, 20 kPa, 10 kPa, or 1 kPa; from 1 kPa to 1 MPa, 100 kPa, 50 kPa, 20 kPa, or 10 kPa; from 10 kPa to 1 MPa, 100 kPa, 50 kPa, or 20 kPa; from 20 kPa to 1 MPa, 100 kPa, or 50 kPa; from 50 kPa to 1 MPa, or 100 kPa; or from 100 kPa to 1 MPa.

In some embodiments, the compositions provided herein have an extension to break of greater than 1%, greater than 10%, greater than 50%, greater than 100%, greater than 300%, or greater than 500%; from 1% to 500%, 300%, 100%, 50%, or 10%; from 10% to 500%, 300%, 100%, or 50%; from 50% to 500%, 300%, or 100%; from 100% to 500%, or 300%; or from 300% to 500%.

In some embodiments, the compositions provided herein have a maximum tensile strength of greater than 0.1 kPa, greater than 1 kPa, greater than 2 kPa, greater than 5 kPa, or greater than 10 kPa; from 0.1 kPa to 100 kPa, 10 kPa, 5 kPa, 2 kPa, or 1 kPa; from 1 kPa to 100 kPa, 10 kPa, 5 kPa, or 2 kPa; from 2 kPa to 100 kPa, 10 kPa, or 5 kPa; from 5 kPa to 100 kPa, or 10 kPa; or from 10 kPa to 100 kPa.

In some embodiments, mechanical properties such as hardness and compressive elastic modulus can be measured using indentation and nanoindentation measurement systems. In some embodiments, indentation measurements utilizing a tip to indent the sample to a given amount of strain are used to measure the hardness and compressive elastic modulus of resilin, and the resulting force is measured using a load cell. In some embodiments, different tip shapes can be used including Vickers and Berkovich shaped tips. In some embodiments, the hardness measured by indentation techniques is characterized by the relation, Hardness=(Peak Force)/(Contact Area).

In some embodiments, the hardness in polymers, elastomers, and rubbers can be measured using a durometer. In some embodiments the hardness of an elastomer can be measured using the standard ASTM D2240, which recognizes twelve different durometer scales using combinations of specific spring forces and indentor configurations. The most common scales are the Shore OO, A and D Hardness Scales. Hardness scales range from 0 to 100, where 0 is softer material and 100 is harder material.

In some embodiments, the compositions provided herein have a Shore 00 Hardness of less than 90, less than 80, less than 70, less than 60, less than 50, less than 40, less than 30, or less than 20; from 10 to 90, 80, 70, 60, 50, 40, 30, or 20; from 20 to 90, 80, 70, 60, 50, 40, or 30; from 30 to 90, 80, 70, 60, 50, or 40; from 40 to 90, 80, 70, 60, or 50; from 50 to 90, 80, 70, or 60; from 60 to 90, 80, or 70; from 70 to 90, or 80; or from 80 to 90. In some embodiments, hardness measurements in resilin are performed according to ASTM D2240.

As used here, the term “rebound” refers to a particular measure of resilience. In some embodiments, rebound can be measured with a number of different tools including pendulum tools and dropped balls. In the pendulum type measurements, RB, commonly called percentage rebound, is determined from the equation:

$$RB = \frac{[ 1 - cos ( angle of rebound ) ]}{[ 1 - cos ( original angle ) ]} \times 100$$

The rebound resilience can be calculated as:

$$R = \frac{h}{H}$$

where h=apex height of the rebound, and H=initial height. The rebound resilience can also be determined by the measurement of the angle of rebound. Some examples of test methods for determining rebound in elastomers are ASTM D2632-15 and ASTM D7121-05(2012).

In some embodiments, the compositions provided herein have a rebound greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%. In some embodiments, rebound measurements in resilin are performed according to ASTM D2632-15, or ASTM D7121-05(2012).

As used herein, the term “compression set” refers to a measure of the permanent deformation remaining after an applied force is removed. In some embodiments, compression set can be measured in different ways, including compression set under constant force in air (referred to as Compression Set A), compression set under constant deflection in air (referred to as Compression Set B), and compression set under constant deflection in air considering material hardness (referred to as Compression Set C). Compression Set A (C_{A}) is calculated by the following expression: C_{A}=[(tₒ−tᵢ)/tₒ]×100, where tₒ is the original specimen thickness, and tᵢ is the specimen thickness after testing). Compression set B (C_{B}) is given by C_{B}=[(tₒ−tᵢ)/(tₒ−tₙ)]×100, where tₒ is the original specimen thickness, tᵢ is the specimen thickness after testing, and tₙ is the spacer thickness or the specimen thickness during the test. Some examples of test methods for determining compression set in elastomers are ASTM D3574-11 and ASTM D395-16.

In some embodiments, the compositions provided herein have a Compression Set A or a Compression Set B of greater than 50%, greater than 60%, greater than 70%, greater than 80%, greater than 90%, or greater than 95%; from 50% to 100%, 90%, 80%, 70%, or 60%; from 60% to 100%, 90%, 80%, or 70%; from 70% to 100%, 90%, or 80%; from 80% to 100%, or 90%; from 90% to 100%; from 95% to 100%, from 90% to 99%, or from 95% to 99%. In some embodiments, compression set measurements in resilin are performed according to ASTM D3574-11 and ASTM D395-16.

The processing and forming of resilin into products can take many forms for different applications. Accordingly, the compositions provided herein can have any shape and form, including but not limited to gels, porous sponges, films, machinable solids, cast forms, molded forms, and composites.

The compositions provided herein have a number of uses, including but not limited to applications in aerospace, automotive, sporting equipment, vibration isolation, footwear, and clothing among others. Some applications from these categories are listed as non-limiting examples. Due to the desirable elastic efficiency, resilin can be used as an energy storage device (e.g., a rubber band) for storing and recovering mechanical energy. Automobile suspension systems can be improved by application of resilin bushings to keep more tire contact on the road when going over bumps and through potholes at speed. Additionally, there are a number of sporting equipment applications for resilin with differently tuned mechanical properties including cores of golf balls, tennis racket grips, golf club grips, and table tennis paddles.

An application of particular interest is footwear due to the unique properties of resilin compositions provided herein. As an insole or midsole, resilin can improve the comfort and bioefficiency of shoes by cushioning the foot strike and restoring more of the energy from that footstrike as forward momentum. As a midsole, resilin can make up the entire midsole or be encapsulated within another material to complement its properties (e.g., an abrasion or wear resistant material, or a material tuned for traction). The resilin midsole can also contain a plurality of resilin materials with differently tuned mechanical properties that work in concert to provide enhanced performance (e.g., softer heel strike area and firmer arch support).

As used herein, the term “density” refers to the mass of the sample divided by the volume. In some embodiments, the density of an elastomer can be determined using a pycnometer with alcohol in place of water to eliminate air bubbles. In some embodiments, the density of an elastomer can be determined using a hydrostatic method. As used herein, the term “compressed volume density” refers to the ratio of the sample mass to the compressed volume of the sample, where the “compressed volume” is defined as the final equilibrium volume attained by an elastomeric sample when it is subjected to a compressive force sufficient to cause it to flow until it fully conforms to the surrounding shape of the piston-cylinder test chamber enclosure. In some embodiments, the compressed volume density of an elastomer can be determined using a compressed volume densimeter.

In some embodiments, the compositions provided herein have a density or a compressed volume density are from 0.5 mg/cm³ to 2.0 mg/cm³, or from 1.0 mg/cm³ to 1.5 mg/cm³, or from 1.1 mg/cm³ to 1.4 mg/cm³, or from 1.2 mg/cm³ to 1.35 mg/cm³. In some embodiments, the determination of the density or the compressed volume density of elastomers can be performed using ASTM D297-15 Standard Test Methods for Rubber Products Chemical Analysis.

### Recombinant Resilin Vectors, Recombinantn Host Cells, and Fermentations

Further provided herein are vectors encoding recombinant resilins, recombinant host cells comprising such vectors, and fermentations comprising such recombinant host cells and recombinant resilins.

In some embodiments, the vectors provided herein comprise secreted resilin coding sequences, which encode a resilin polypeptide fused at its N-terminus tₒ a secretion signal and optionally at its C-terminus tₒ a tag peptide or polypeptide. In some embodiments, the vectors comprise secreted resilin coding sequences that are codon-optimized for expression in a particular host cell.

Suitable secretion signals are secretion signals that mediate secretion of polypeptides in the recombinant host cells provided herein. Non-limiting examples of suitable secretion signals are the secretion signals of the alpha mating factor (α-MF) of Saccharomyces cerevisiae, acid phosphatase (PHO1) of Pichia pastoris, and phytohemagglutinin (PHA-E) from the common bean Phaseolus vulgaris. Additional secretion signals are known in the art, or can be identified by identification of proteins secreted by a host cell followed by genomic analysis of the secreted proteins and identification of the non-translated N-terminal sequences (see, for example, Huang et al. A proteomic analysis of the Pichia pastoris secretome in methanol-induced cultures. Appl Microbiol Biotechnol. 2011 April; 90(1):235-47).

The resilins encoded by the secreted resilin coding sequences can be further fused to tag peptides or polypeptides. Non-limiting examples of tag peptides or polypeptides include affinity tags (i.e., peptides or polypeptides that bind to certain agents or matrices), solubilization tags (i.e., peptides or polypeptides that assist in proper folding of proteins and prevent precipitation), chromatography tags (i.e., peptides or polypeptides that alter the chromatographic properties of a protein to afford different resolution across a particular separation techniques), epitope tags (i.e., peptides or polypeptides that are bound by antibodies), fluorescence tags (i.e., peptides or polypeptides that upon excitation with short-wavelength light emit high-wavelength light), chromogenic tags (i.e., peptides or polypeptides that absorb specific segments of the visible light spectrum), enzyme substrate tags (i.e., peptides or polypeptides that are the substrates for specific enzymatic reactions), chemical substrate tags (i.e., peptides or polypeptides that are the substrates for specific chemical modifications), or combinations thereof. Non-limiting examples of suitable affinity tags include maltose binding protein (MBP), glutathione-S-transferase (GST), poly(His) tag, SBP-tag, Strep-tag, and calmodulin-tag. Non-limiting examples of suitable solubility tags include thioredoxin (TRX), poly(NANP), MBP, and GST. Non-limiting examples of chromatography tags include polyanionic amino acids (e.g., FLAG-tag [GDYKDDDDKDYKDDDDKDYKDDDDK (SEQ ID NO: 45)]) and polyglutamate tag. Non-limiting examples of epitope tags include V5-tag, VSV-tag, Myc-tag, HA-tag, E-tag, NE-tag, and FLAG-tag. Non-limiting examples of fluorescence tags include green fluorescent protein (GFP), blue fluorescent protein (BFP), cyan fluorescent protein (CFP), yellow fluorescent protein (YFP), orange fluorescent protein (OFP), red fluorescent protein (RFP), and derivatives thereof. Non-limiting examples of chromogenic tags include non-fluorescent members of the GFP-like family of proteins (e.g., BlitzenBlue, DonnerMagenta; DNA2.0, Neward, CA). Non-limiting examples of enzyme substrate tags include peptides or polypeptides comprising a lysine within a sequence suitable for biotinilation (e.g., AviTag, Biotin Carboxyl Carrier Protein [BCCP]). Non-limiting examples of chemical substrate tags include substrates suitable for reaction with FIASH-EDT2. The fusion of the C-terminal peptide or polypeptide to the resilin can be cleavable (e.g., by TEV protease, thrombin, factor Xa, or enteropeptidase) on non-cleavable.

In some embodiments, the vectors comprise single secreted resilin coding sequences. In other embodiments, the vectors comprise 2 or more (e.g., 3, 4, or 5) secreted resilin coding sequences. In some such embodiments, the secreted resilin coding sequences are identical. In other such embodiments, at least 2 of the secreted resilin coding sequences are not identical. In embodiments in which at least 2 of the secreted resilin coding sequences are not identical, the at least 2 secreted resilin coding sequences can differ from each other in the resilins and/or in the secretion signals and/or the optional tag peptides or polypeptides they encode.

In some embodiments, the vectors comprise promoters that are operably linked to the secreted resilin coding sequences such that they drive the expression of the secreted resilin coding sequences. The promotors can be constitutive promoters or inducible promoters. In some embodiments, induction of the inducible promoter occurs via glucose repression, galactose induction, sucrose induction, phosphate repression, thiamine repression, or methanol induction. Suitable promoters include promoters that mediate expression of proteins in the recombinant host cells provided herein. Non-limiting examples of suitable promoters include the AOX1 promoter, GAP promoter, LAC4-PBI promoter, T7 promoter, TAC promoter, GCW14 promoter, GAL1 promoter, λPL promoter, λPR promoter, beta-lactamase promoter, spa promoter, CYC1 promoter, TDH3 promoter, GPD promoter, TEF1 promoter, ENO2 promoter, PGL1 promoter, SUC2 promoter, ADH1 promoter, ADH2 promoter, HXT7 promoter, PHO5 promoter, and CLB1 promoter. Additional promoters that can be used to facilitate expression of the secreted resilin coding sequences are known in the art.

In some embodiments, the vectors comprise terminators that are operably linked to the secreted resilin coding sequences such that they effect termination of transcription of the secreted resilin coding sequences. Suitable terminators include terminators that terminate transcription in the recombinant host cells provided herein. Non-limiting examples of suitable terminators include the AOX1 terminator, PGK1 terminator, and TPS1 terminator. Additional terminators that effect termination of transcription of the secreted resilin coding sequences are known in the art.

In embodiments in which the vectors comprise 2 or more resilin coding sequences, the 2 or more resilin coding sequences can be operably linked to the same promoters and/or terminators or to 2 or more different promoters and/or terminators.

The vectors provided herein can further comprise elements suitable for propagation of the vectors in recombinant host cells. Non-limiting examples of such elements include bacterial origins of replication and selection markers (e.g., antibiotic resistance genes, auxotrophic markers). Bacterial origins of replication and selection markers are known in the art. In some embodiments, the selection marker is a drug resistant marker. A drug resistant maker enables cells to detoxify an exogenously added drug that would otherwise kill the cell. Illustrative examples of drug resistant markers include but are not limited to those for resistance to antibiotics such as ampicillin, tetracycline, kanamycin, bleomycin, streptomycin, hygromycin, neomycin, Zeocin™, and the like. In some embodiments, the selection marker is an auxotrophic marker. An auxotrophic marker allows cells to synthesize an essential component (usually an amino acid) while grown in media that lacks that essential component. Selectable auxotrophic gene sequences include, for example, hisD, which allows growth in histidine-free media in the presence of histidinol. Other selection markers suitable for the vectors of the present invention include a bleomycin-resistance gene, a metallothionein gene, a hygromycin B-phosphotransferase gene, the AURI gene, an adenosine deaminase gene, an aminoglycoside phosphotransferase gene, a dihydrofolate reductase gene, a thymidine kinase gene, and a xanthine-guanine phosphoribosyltransferase gene.

The vectors of the present invention can further comprise targeting sequences that direct integration of the secreted resilin coding sequences to specific locations in the genome of host cells. Non-limiting examples of such targeting sequences include nucleotide sequences that are identical to nucleotide sequences present in the genome of a host cell. In some embodiments, the targeting sequences are identical to repetitive elements in the genome of host cells. In some embodiments, the targeting sequences are identical to transposable elements in the genome of host cells.

In some embodiments, recombinant host cells are provided herein that comprise the vectors described herein. In some embodiments, the vectors are stably integrated within the genome (e.g., a chromosome) of the recombinant host cells, e.g., via homologous recombination or targeted integration. Non-limiting examples of suitable sites for genomic integration include the Tyl loci in the Saccharomyces cerevisiae genome, the rDNA and HSP82 loci in the Pichia pastoris genome, and transposable elements that have copies scattered throughout the genome of the recombinant host cells. In other embodiments, the vectors are not stably integrated within the genome of the recombinant host cells but rather are extrachromosomal.

Recombinant host cells can be of mammalian, plant, algae, fungi, or microbe origin. Non-limiting examples of suitable fungi include methylotrophic yeast, filamentous yeast, Arxula adeninivorans, Aspergillus niger, Aspergillus niger var. awamori, Aspergillus oryzae, Candida etchellsii, Candida guilliermondii, Candida humilis, Candida lipolytica, Candida pseudotropicalis, Candida utilis, Candida versatilis, Debaryomyces hansenii, Endothia parasitica, Eremothecium ashbyii, Fusarium moniliforme, Hansenula polymorpha, Kluyveromyces lactis, Kluyveromyces marxianus, Kluyveromyces thermotolerans, Morteirella vinaceae var. raffinoseutilizer, Mucor miehei, Mucor miehei var. Cooney et Emerson, Mucor pusillus Lindt, Penicillium roquefortii, Pichia methanolica, Pichia pastoris (Komagataella phaffii), Pichia (Scheffersomyces) stipitis, Rhizopus niveus, Rhodotorula sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum, Sporidiobolus johnsonii, Sporidiobolus salmonicolor, Sporobolomyces roseus, Trichoderma reesi, Xanthophyllomyces dendrorhous, Yarrowia lipolytica, Zygosaccharomyces rouxii, and derivatives and crosses thereof.

Non-limiting examples of suitable microbes include Acetobacter suboxydans, Acetobacter xylinum, Actinoplane missouriensis, Arthrospira platensis, Arthrospira maxima, Bacillus cereus, Bacillus coagulans, Bacillus licheniformis, Bacillus stearothermophilus, Bacillus subtilis, Escherichia coli, Lactobacillus acidophilus, Lactobacillus bulgaricus, Lactobacillus reuteri, Lactococcus lactis, Lactococcus lactis Lancefield Group N, Leuconostoc citrovorum, Leuconostoc dextranicum, Leuconostoc mesenteroides strain NRRL B-512(F), Micrococcus lysodeikticus, Spirulina, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, Xanthomonas campestris, and derivatives and crosses thereof. Additional strains that can be used as recombinant host cells are known in the art. It should be understood that the term “recombinant host cell” is intended to refer not only to the particular subject cell but to the progeny of such a cell. Because certain modifications may occur in succeeding generations due to either mutation or environmental influences, such progeny may not, in fact, be identical to the parent cell, but is still included within the scope of the term “recombinant host cell” as used herein.

In some embodiments, the recombinant host cells comprise genetic modifications that improve production of the recombinant resilins provided herein. Non-limiting examples of such genetic modifications include altered promoters, altered kinase activities, altered protein folding activities, altered protein secretion activities, altered gene expression induction pathways, and altered protease activities.

The recombinant host cells provided herein are generated by transforming cells of suitable origin with vectors provided herein. For such transformation, the vectors can be circularized or be linear. Recombinant host cell transformants comprising the vectors can be readily identified, e.g., by virtue of expressing drug resistance or auxotrophic markers encoded by the vectors that permit selection for or against growth of cells, or by other means (e.g., detection of light emitting peptide comprised in vectors, molecular analysis of individual recombinant host cell colonies, e.g., by restriction enzyme mapping, PCR amplification, or sequence analysis of isolated extrachromosomal vectors or chromosomal integration sites).

In some embodiments, the recombinant host cells provided herein can produce high titers of the recombinant resilins provided herein. In some such embodiments, the recombinant host cells produce the recombinant resilins at a rate of greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. In other such embodiments, the recombinant host cells secrete the recombinant resilins at a rate of greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. The identities of the recombinant resilins produced can be confirmed by HPLC quantification, Western blot analysis, polyacrylamide gel electrophoresis, and 2-dimensional mass spectroscopy (2D-MS/MS) sequence identification.

In some embodiments, the recombinant host cells provided herein have high secreted fractions of the recombinant resilins provided herein. In some such embodiments, the recombinant host cells have secreted fractions of recombinant resilient that is greater than 50%, 60%, 70%, 80%, or 90%; from 50% to 100%, 90%, 80%, 70%, or 60%; from 60% to 100%, 90%, 80%, or 70%; from 70% to 100%, 90%, or 80%; from 90% to 100%, or 90%; or from 90% to 100%.

Production and secretion of recombinant resilins can be influenced by the number of copies of the secreted resilin coding sequences comprised in the recombinant host cells and/or the rate of transcription of the secreted resilin coding sequences comprised in the recombinant host cells. In some embodiments, the recombinant host cells comprise a single secreted resilin coding sequence. In other embodiments, the recombinant host cells comprise 2 or more (e.g., 3, 4, 5, or more) secreted resilin coding sequences. In some embodiments, the recombinant host cells comprise secreted resilin coding sequences that are operably linked to strong promoters. Non-limiting examples of strong promoters include the pGCW14 promoter of Pichia pastoris. In some embodiments, the recombinant host cells comprise secreted resilin coding sequences that are operably linked to medium promoters. Non-limiting examples of such medium promoters include the pGAP promoter of Pichia pastoris. In some embodiments, the recombinant host cells comprise coding sequences encoding resilins under the control of weak promoters.

The fermentations provided herein comprise recombinant host cells described herein and a culture medium suitable for growing the recombinant host cells.

The fermentations are obtained by culturing the recombinant host cells in culture media that provide nutrients needed by the recombinant host cells for cell survival and/or growth, and for secretion of the recombinant resilins. Such culture media typically contain an excess carbon source. Non-limiting examples of suitable carbon sources include monosaccharides, disaccharides, polysaccharides, and combinations thereof. Non-limiting examples of suitable monosaccharides include glucose, galactose, mannose, fructose, ribose, xylose, arabinose, ribose, and combinations thereof. Non-limiting examples of suitable disaccharides include sucrose, lactose, maltose, trehalose, cellobiose, and combinations thereof. Non-limiting examples of suitable polysaccharides include raffinose, starch, glycogen, glycan, cellulose, chitin, and combinations thereof.

In some embodiments, the fermentations comprise recombinant resilins in amounts of at least 1%, 5%, 10%, 20%, or 30%; from 1% to 100%, 90%, 80%, 70%, 60%, 50%, 40%, 30%, 20%, or 10%; from 10% to 100%, 90%, 80%, 70%, 60%, 50%, 40%, 30%, or 20%; from 20% to 100%, 90%, 80%, 70%, 60%, 50%, 40%, or 30%; from 30% to 100%, 90%, 80%, 70%, 60%, 50%, or 40%; from 40% to 100%, 90%, 80%, 70%, 60%, or 50%; from 50% to 100%, 90%, 80%, 70%, or 60%; from 60% to 100%, 90%, 80%, or 70%; from 70% to 100%, 90%, or 80%; from 80% to 100%, or 90%; or from 90% to 100% by weight of the total fermentation.

In some embodiments, the fermentations comprise recombinant resilin in an amount of at least 2 g/L, 5 g/L, 10 g/L, 15 g/L, 20 g/L, 25 g/L, or 30 g/L; from 2 g/L to 300 g/L, 200 g/L, 100 g/L, 90 g/L, 80 g/L, 70 g/L, 60 g/L, 50 g/L, 40 g/L, 30 g/L, 20 g/L, or 10 g/L; from 10 g/L to 300 g/L, 200 g/L, 100 g/L, 90 g/L, 80 g/L, 70 g/L, 60 g/L, 50 g/L, 40 g/L, 30 g/L, or 20 g/L; from 20 g/L to 300 g/L, 200 g/L, 100 g/L, 90 g/L, 80 g/L, 70 g/L, 60 g/L, 50 g/L, 40 g/L, or 30 g/L; from 30 g/L to 300 g/L, 200 g/L, 100 g/L, 90 g/L, 80 g/L, 70 g/L, 60 g/L, 50 g/L, or 40 g/L; from 40 g/L to 300 g/L, 200 g/L, 100 g/L, 90 g/L, 80 g/L, 70 g/L, 60 g/L, or 50 g/L; from 50 g/L to 300 g/L, 200 g/L, 100 g/L, 90 g/L, 80 g/L, 70 g/L, or 60 g/L; from 60 g/L to 300 g/L, 200 g/L, 100 g/L, 90 g/L, 80 g/L, or 70 g/L; from 70 g/L to 300 g/L, 200 g/L, 100 g/L, 90 g/L, or 80 g/L; from 80 g/L to 300 g/L, 200 g/L, 100 g/L, or 90 g/L; from 90 g/L to 300 g/L, 200 g/L, or 100 g/L; from 100 g/L to 300 g/L, or 200 g/L; or from 200 g/L to 300 g/L.

### METHODS

Further provided herein are methods for the production of the recombinant resilins described herein.

The methods are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y., 1989; Ausubel et al., Current Protocols in Molecular Biology, Greene Publishing Associates, 1992, and Supplements to 2002); Harlow and Lane, Antibodies: A Laboratory Manual, Cold Spring Harbor Laboratory Press, Cold Spring Harbor, N.Y., 1990; Taylor and Drickamer, Introduction to Glycobiology, Oxford Univ. Press, 2003; Worthington Enzyme Manual, Worthington Biochemical Corp., Freehold, N.J.; Handbook of Biochemistry: Section A Proteins, Vol I, CRC Press, 1976; Handbook of Biochemistry: Section A Proteins, Vol II, CRC Press, 1976; Essentials of Glycobiology, Cold Spring Harbor Laboratory Press, 1999.

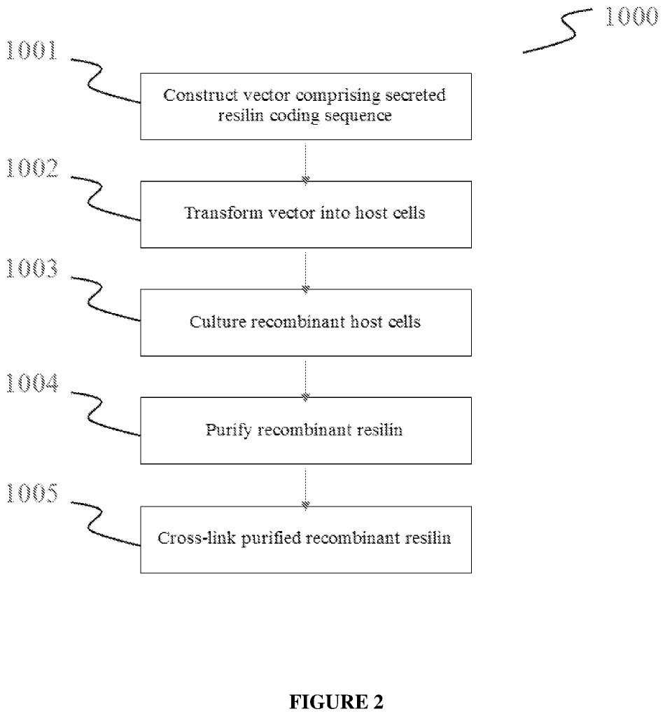

In some embodiments, a novel method is utilized to secrete resilin extracellularly from a host cell. In some embodiments, the method comprises constructing a vector comprising a secreted resilin coding sequence (step 1001 in FIG. 2), transforming the vector into a host cell (step 1002 in FIG. 2), and then culturing the recombinant host cells to secrete resilin extracellularly (step 1003 in FIG. 2). In some embodiments, the method includes secreting the resilin extracellularly at a rate greater than 2 mg resilin/g dry cell weight/hour, 4 mg resilin/g dry cell weight/hour, 6 mg resilin/g dry cell weight/hour, 8 mg resilin/g dry cell weight/hour, 10 mg resilin/g dry cell weight/hour, 12 mg resilin/g dry cell weight/hour, 14 mg resilin/g dry cell weight/hour, 16 mg resilin/g dry cell weight/hour, 18 mg resilin/g dry cell weight/hour, 20 mg resilin/g dry cell weight/hour, 25 mg resilin/g dry cell weight/hour, or 30 mg resilin/g dry cell weight/hour; from 2 to 40, 30, 20, 10, or 5 mg resilin/g dry cell weight/hour; from 5 to 40, 30, 20, or 10 mg resilin/g dry cell weight/hour; from 10 to 40, 30, or 20 mg resilin/g dry cell weight/hour; from 20 to 40, or 30 mg resilin/g dry cell weight/hour; or from 30 to 40 mg resilin/g dry cell weight/hour. In some embodiments, the secreted resilin is then purified (step 1004 in FIG. 2), and the purified resilin is cross-linked to form an elastomer (step 1005 in FIG. 2). In some embodiments, the methods provided herein comprise the step of transforming cells with vectors provided herein to obtain recombinant host cells provided herein (step 1002 in FIG. 2). Methods for transforming cells with vectors are well-known in the art. Non-limiting examples of such methods include calcium phosphate transfection, dendrimer transfection, liposome transfection (e.g., cationic liposome transfection), cationic polymer transfection, electroporation, cell squeezing, sonoporation, optical transfection, protoplast fusion, impalefection, hyrodynamic delivery, gene gun, magnetofection, and viral transduction. One skilled in the art is able to select one or more suitable methods for transforming cells with vectors provided herein based on the knowledge in the art that certain techniques for introducing vectors work better for certain types of cells.

In some embodiments, the methods further comprise the step of culturing the recombinant host cells provided herein in culture media under conditions suitable for obtaining the fermentations provided herein (step 1003 in FIG. 2). In some embodiments, the conditions and culture media are suitable to facilitate secretion of the recombinant proteins from the recombinant host cells into the culture media. Suitable culture media for use in these methods are known in the art, as are suitable culture conditions. Exemplary details of culturing yeast host cells are described in Idiris et al., Appl. Microbiol. Biotechnol. 86:403-417, 2010; Zhang et al., Biotechnol. Bioprocess. Eng. 5:275-287, 2000; Zhu, Biotechnol. Adv. 30:1158-1170, 2012; Li et al., MAbs 2:466-477, 2010.

In some embodiments, the methods further comprise the step of purifying secreted recombinant resilins from the fermentations provided herein to obtain the recombinant resilins provided herein (step 1004 in FIG. 2). Purification can occur by a variety of methods known in the art for purifying secreted proteins from fermentations. Common steps in such methods include centrifugation (to remove cells) followed by precipitation of the proteins using precipitants or other suitable cosmotropes (e.g., ammonium sulfate). The precipitated protein can then be separated from the supernatant by centrifugation, and resuspended in a solvent (e.g., phosphate buffered saline [PBS]). The suspended protein can be dialyzed to remove the dissolved salts. Additionally, the dialyzed protein can be heated to denature other proteins, and the denatured proteins can be removed by centrifugation. Optionally, the purified recombinant resilins can be coacervated.

In various embodiments, methods of purifying the secreted recombinant proteins from the fermentation can include various centrifugation steps in conjunction with solubilizing protein in a whole cell broth or cell pellet with known chaotropes such as urea or guanidine thiocyanate.

In some embodiments, the methods provided herein further comprise the step of cross-linking the recombinant resilins to obtain the recombinant resilin compositions provided herein (step 1005 in FIG. 2). Methods for cross-linking proteins are known in the art. In some embodiments, cross-linking is achieved via enzymatic cross-linking (e.g., using horseradish peroxidase). In other embodiments, cross-linking is achieved via photochemical cross-linking (see, for example, Elvin CM, Carr AG, Huson MG, Maxwell JM, Pearson RD, Vuocolo T, Liyou NE, Wong DCC, Merritt DJ, Dixon NE. Nature 2005, 437, 999-1002; Whittaker JL, Dutta NK, Elvin CM, Choudhury NR. Journal of Materials Chemistry B 2015, 3, 6576-79; Degtyar E, Mlynarczyk B, Fratzl P, Harrington MJ. Polymer 2015, 69, 255-63). In some embodiments, cross-linking is achieved via chemical cross-linking (see, for example, Renner JN, Cherry KM, Su RSC, Liu JC. Biomacromolecules 2012, 13, 3678-85; Charanti, MB, Ifkovits, JL, Burdick, JA, Linhardt JG, Kiick, KL. Soft Matter 2009, 5, 3412-16; Li LQ, Tong ZX, Jia XQ, Kiick KL. Soft Matter 2013, 9, 665-73; Li L, Mahara A, Tong Z, Levenson EA, McGann CL, Jia X, Yamaoka T, Kiick KL. Advanced Healthcare Materials 2016, 5, 266-75). In some embodiments, cross-linking is achieved via tyrosine residues. In other embodiments, cross linking is achieved via lysine residues. In some embodiments, cross linking is achieved via cysteine residues. In some embodiments, cross-linking employs transglutaminase (see, for example, Kim Y, Gill EE, Liu JC. Enzymatic Cross-Linking of Resilin-Based Proteins for Vascular Tissue Engineering Applications. Biomacromolecules. 17(8):2530-9). In some embodiments, cross-linking employs poly(ethylene glycol) (PEG) (McGann CL, Levenson EA, Kiick KL. Macromol. Chem. Phys. 2013, 214, 203-13; McGann CL, Akins RE, Kiick KL. Resilin-PEG Hybrid Hydrogels Yield Degradable Elastomeric Scaffolds with Heterogeneous Microstructure. Biomacromolecules. 2016; 17(1): 128-40). In some embodiments, cross-linking occurs in vessels or molds such that the recombinant resilin compositions obtained have specific shapes or forms.

### EXAMPLES

### Example 1: Generation of Pichia pastoris Recombinant Host Cells that Secrete Recombinant Resilin

Pichia pastoris recombinant host cells that secrete recombinant resilin were generated by transforming a HIS+ derivative of GS115 (NRRL Y15851) Pichia pastoris (Komagataella phaffii) with vectors comprising secreted resilin coding sequences.

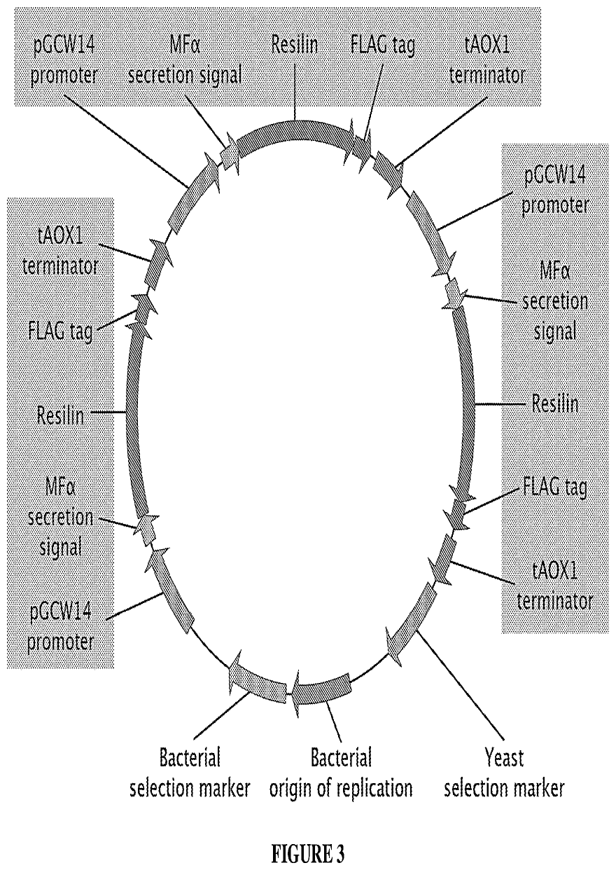

The vectors each comprised 3 resilin coding sequences fused in frame to an N-terminal secretion signal (alpha mating factor leader and pro sequence), and in some instances a C-terminal 3xFLAG tag (SEQ ID NO: 45) (see FIG. 3). Each of the secreted resilin coding sequences was flanked by a promoter (pGCW14) and a terminator (tAOX1 pA signal). The vectors further comprised a targeting region that can direct integration of the 3 secreted resilin coding sequences to the HSP82 locus of the Pichia pastoris genome, dominant resistance markers for selection of bacterial and yeast transformants, as well as a bacterial origin of replication.

The resilin coding sequences were obtained from scientific literature and from searching public sequence databases. The nucleotide sequences were translated into amino acid sequences and then codon-optimized. Both full length and truncated resilin sequences were chosen. Selected secreted resilin coding sequences are listed in Table 1.

TABLE 1
Exemplary full-length and truncated resilin amino acid sequences and recombinant host strains

| | | | Amino | | | | |
|---|---|---|---|---|---|---|---|
| | | | Acid | | | | |
| | | Short | SEQ | With FLAG tag | | Without FLAG tag | |
| Species | Type | Name | ID NO: | Plasmid | Strain | Plasmid | Strain |
| Drosophila | Full length | Ds_ACB | 1 | RMp4830 | RMs1209 | RMp4842 | RMs1221 |
| sechellia | | | | | | | |
| Drosophila | A repeats + | Ds_AC | 2 | RMp4831 | RMs1210 | RMp4843 | RMs1222 |
| sechellia | Chitin | | | | | | |
| | binding | | | | | | |
| | domain | | | | | | |
| Drosophila | A repeats only | Ds_A | 3 | RMp4832 | RMs1211 | RMp4844 | RMs1223 |
| sechellia | | | | | | | |
| Acromyrmex | A repeats only | Ae_A | 4 | RMp4833 | RMs1212 | RMp4845 | RMs1224 |
| echinatior | | | | | | | |
| Aeshna sp. | B repeats only | As_B | 5 | RMp4834 | RMs1213 | RMp4846 | RMs1225 |
| Aeshna sp. | Full length | As_ACB | 6 | RMp4835 | RMs1214 | RMp4847 | RMs1226 |
| Haematobia | A repeats only | Hi_A | 7 | RMp4836 | RMs1215 | RMp4848 | RMs1227 |
| irritans | | | | | | | |
| Haematobia | Full length | Hi_ACB | 8 | RMp4837 | RMs1216 | RMp4849 | RMs1228 |
| irritans | | | | | | | |
| Ctenocephalides | A repeats only | Cf_A | 9 | RMp4838 | RMs1217 | RMp4850 | RMs1229 |
| felis | | | | | | | |
| Ctenocephalides | B repeats only | Cf_B | 10 | RMp4839 | RMs1218 | RMp4851 | RMs1230 |
| felis | | | | | | | |
| Bombus | A repeats only | Bt_A | 11 | RMp4840 | RMs1219 | RMp4852 | RMs1231 |
| terrestris | | | | | | | |
| Tribolium | A repeats only | Tc_A | 12 | RMp4841 | RMs1220 | RMp4853 | RMs1232 |
| castaneum | | | | | | | |

The vectors were transformed into Pichia pastoris using electroporation to generate host strains comprising 3 integrated copies of each secreted resilin coding sequence. Transformants were plated on YPD agar plates supplemented with an antibiotic, and incubated for 48 hours at 30° C.

Clones from each final transformation were inoculated into 400 μL of Buffered Glycerol-complex Medium (BMGY) in 96-well blocks, and incubated for 24 hours at 30° C. with agitation at 1,000 rpm. A sample was removed, the recombinant host cells were pelleted via centrifugation, and the supernatant was recovered and run on a SDS-PAGE gel for analysis of resilin content via Coomassie gel and Western blot analysis (for polypeptides comprising the 3xFLAG tag). For FLAG-tagged proteins, the remaining cultures were used to inoculate minimal media cultures in duplicate for ELISA measurements. One duplicate was pelleted and the supernatant was measured directly. The second duplicate was extracted with guanidine thiocyanate and both the intra- and extra-cellular fractions were measured.

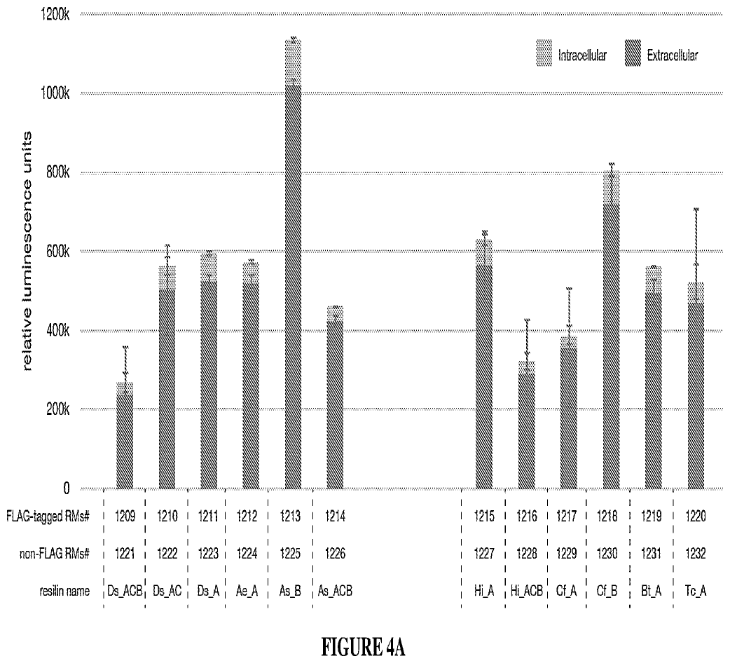

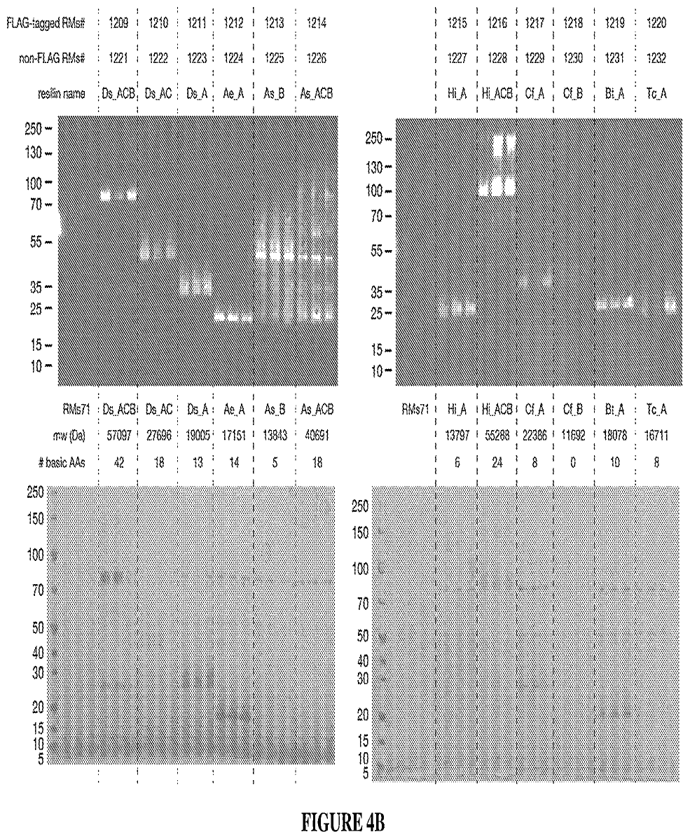

As shown in FIG. 4B and FIG. 4C, recombinant resilin from numerous species expressed successfully in the Pichia pastoris recombinant host cells. (Note: Some proteins have very few basic residues, and are therefore difficult to detect by Coomassie, though they have a signal on Western.) As shown in FIG. 4A, recombinant host cells secreted up to 90% of the recombinant resilin produced.

### Example 2: Measuring Productivity of Pichia pastoris Recombinant Host Cells Expressing and Secreting Recombinant Resilin

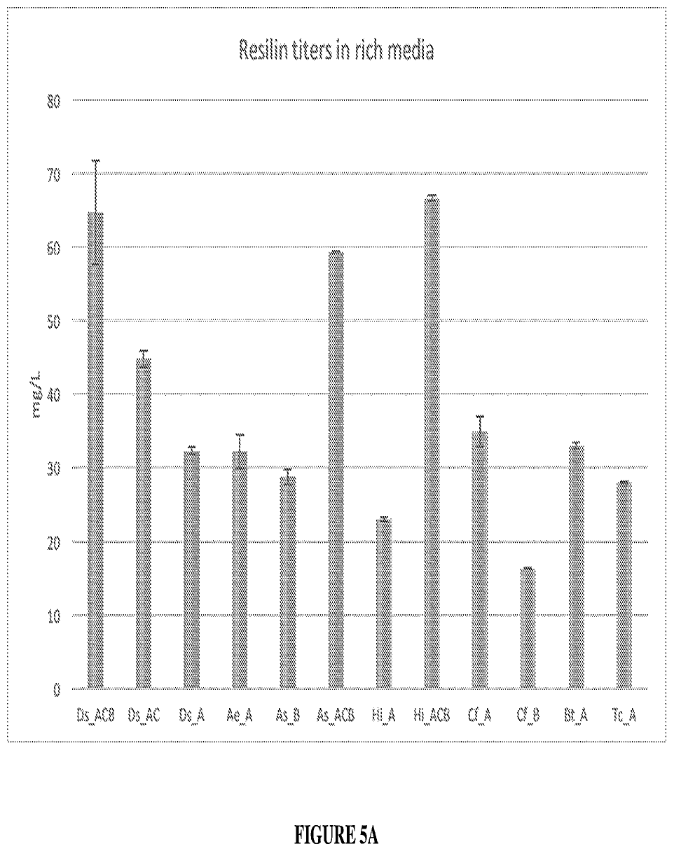

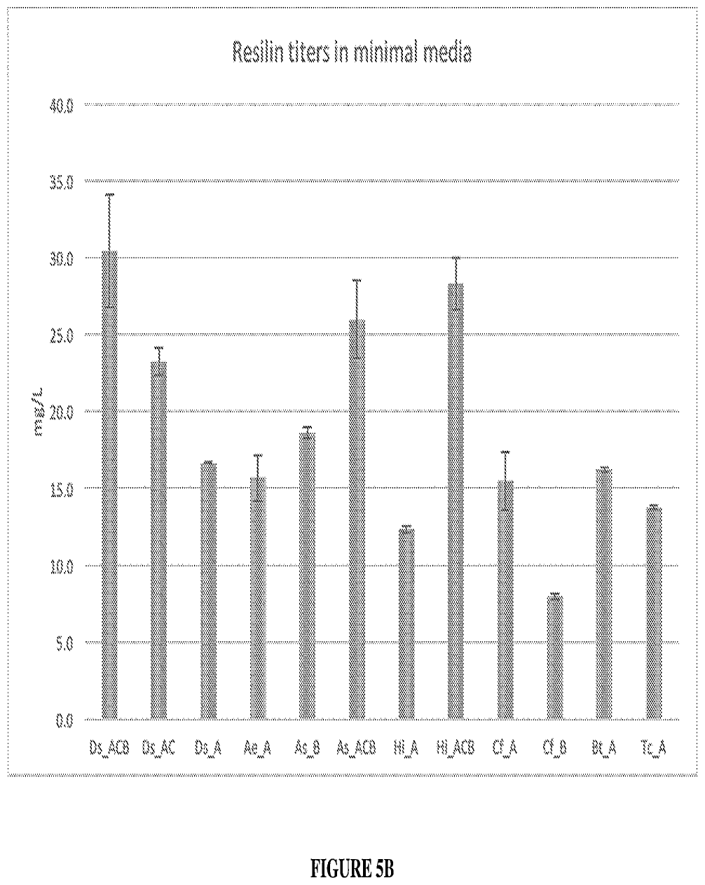

To measure productivity, 3 clones of each recombinant host cell were inoculated into 400 μL of BMGY in a 96-well square-well block, and incubated for 48 hours at 30° C. with agitation at 1,000 rpm. Following the 48-hour incubation, 4 μL of each culture was used to inoculate 400 μL of minimal media in a 96-well square-well block, which was then incubated for 48 hours 30° C. with agitation at 1,000 rpm. 400 μL of 5M guanidine thiocyanate was added to the cultures, and the mixtures were pelleted by centrifugation. The supernatants were saved whereas the pellets were resuspended in 800 μL of 2.5M guanidine thiocyanate. The resuspended cells were physically lysed using beads, the lysed cell mixture was pelleted by centrifugation, and the supernatant was saved. The concentration of resilin in each fraction was determined by direct enzyme-linked immunosorbent assay (ELISA) analysis quantifying the 3xFLAG epitope (FIG. 5A and FIG. 5B).

### Example 3: Purification of Recombinant Resilin

The non-FLAG-tagged Ds_ACB and Ae_A polypeptides were chosen for purification and cross-linking. Strains RMs1221 (expressing Ds_ACB) and RMs1224 (expressing Ae_A) were grown in 500 mL of BMGY in flasks for 48 hours at 30° C. with agitation at 300 rpm.

The protocol for purification was adapted from Lyons et al. (2007). Cells were pelleted by centrifugation, and supernatants were collected. Proteins were precipitated by addition of ammonium sulfate. The precipitated proteins were resuspended in a small volume of phosphate buffered saline (PBS), and the resuspended samples were dialyzed against PBS to remove salts. The dialyzed samples were then heated to denature native proteins, and denatured proteins were removed by centrifugation. The retained supernatants contained the purified resilin polypeptides. Optionally, the retained supernatants were chilled, which caused coacervation, resulting in a concentrated lower phase and dilute upper phase.

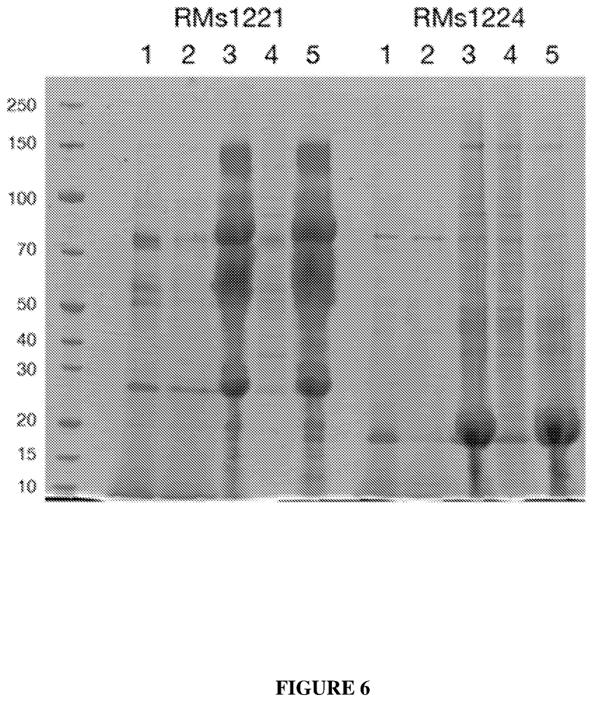

As shown in FIG. 6, Ae_A was obtained in relatively pure form whereas Ds_ACB produced 3 bands at 70 kDa, 50 kDa, and 25 kDa.

### Example 4: Cross-Linking of Purified, Secreted, Recombinant Resilin

Concentrated Ds_ACB resilin was cross-linked via one of two methods: photo cross-linking (adapted from Elvin et al. 2005) and enzymatic cross-linking (adapted from Qin et al. 2009).

For photo cross-linking, resilin protein was mixed with ammonium persulfate and tris (bipyridine) ruthenium (II) ([Ru(bpy)3]2+). The mixture was exposed to bright white light, after which the mixture formed a rubbery solid.

For enzymatic cross-linking, resilin protein was mixed with horseradish peroxidase (HRP) and hydrogen peroxide. The mixture was incubated at 37° C., after which the mixture formed a rubbery solid.

### Example 5: Production of a block of recombinant resilin

Strain RMs1221 (expressing the Ds_ACB resilin) was run in two 2 L fermentation tanks to produce a larger quantity of protein.

The strain was grown in a minimal basal salt media with 15 g/L of glucose as a starting feedstock and 1 g/L L81 antifoam, in a stirred fermentation vessel controlled at 30° C., with 1 VVM of air flow and minimum agitation of 700 rpm. The pH of the fermentation was controlled at 5 with on-demand addition of ammonium hydroxide. Once batch glucose was depleted, glucose was added via a programmed feed recipe that was designed to maintain the oxygen uptake rate 120 mmole/L/h, the temperature was decreased to 25° C., and dissolved oxygen was maintained at 20%. The fermentation was harvested after 70 hours, at about 700-800 OD of cell density.

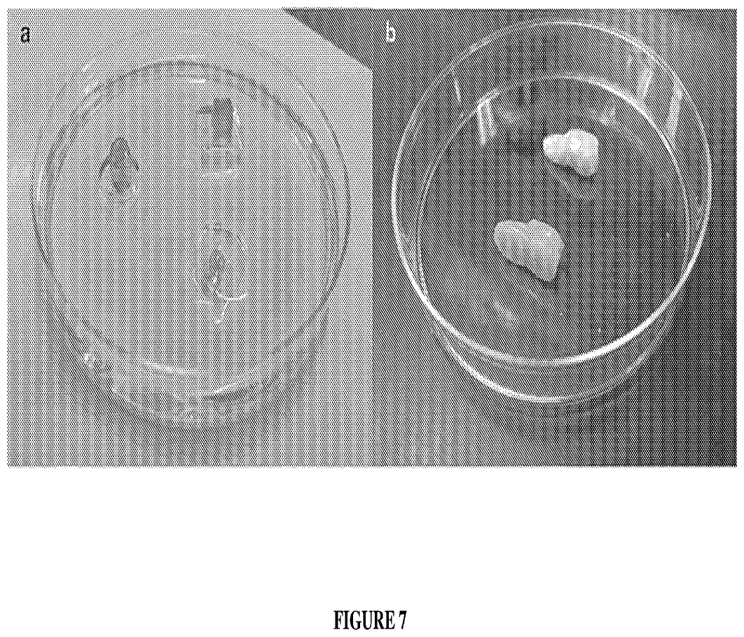

The protein was purified as described in Example 3, and combined with reagents for enzymatic cross-linking as described in Example 4. The cross-linking mixture was filled into small cylindrical, rectangular, spherical, and shoe-shaped molds, and finally incubated at 37° C. Resulting recombinant resilin solids are shown in FIG. 7.

### Example 6: Material Testing of Resilin Solids

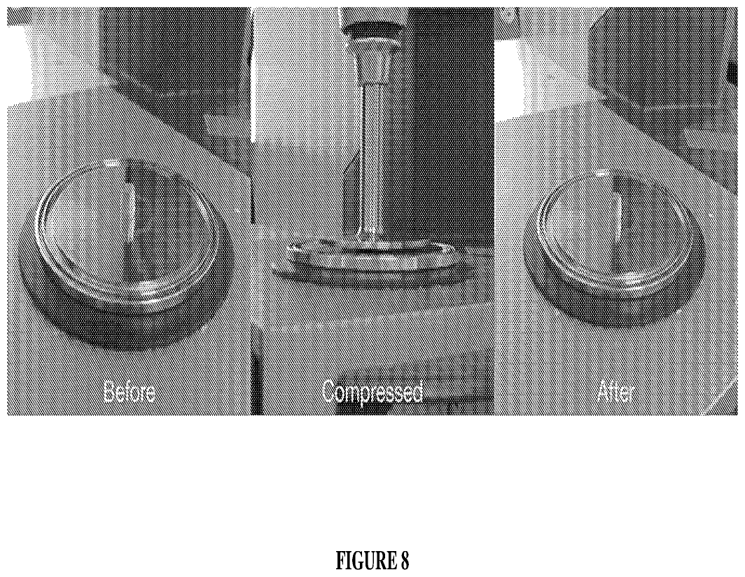

A resilin cylinder produced as described in Example 5 was subjected to a compression test using a rheometer. The recombinant resilin cylinder could be compressed from an initial height of 7.3 mm (avg width 5.4 mm) to less than 0.66 mm without any breakage. As shown in FIG. 8, the cylinder returned to a height of 6.7 mm (avg width 5.6 mm) upon release of the compressive load.

### Example 7: Methods for Recovering Full-Length Recombinant Resilin from Whole Cell Broth

Various recovery and separation techniques were used to purify Ds_ACB (SEQ ID NO: 1) that was produced in strains with a 3X FLAG tag (RMs1209) and without a 3X FLAG tag (RMs1221) according to Example 1 above.

A first set of samples was prepared by centrifuging a whole cell broth to produce a first pellet of cells and a first supernatant, and extracting the first supernatant to produce a clear cell broth. The first supernatant was then precipitated using ammonium sulfate and centrifuged to produce a second pellet and second supernatant which was discarded. The second pellet was then re-suspended in PBS for dialysis. The dialyzed solution was then subject to high temperature to denature proteins other than Ds_ACB, which is stable at high temperatures. The denatured proteins were removed by centrifuging the dialyzed and denatured solution to produce a third pellet and third supernatant. The third supernatant was retained from the denatured solution, then coacervated by chilling the third supernatant to induce a phase separation into a dense lower layer containing the Ds_ACB and an upper layer. These samples are referred to in Table 2 below and elsewhere herein as the “CCB” samples. In some CCB samples, multiple coacervations were performed by retaining the lower layer and incubating the lower layer at a lower temperature to induce further phase separation. These CCB samples are respectively referred to in Table 2 below and elsewhere herein as the “first coacervation” and “second coacervation” samples.

A second set of samples was prepared by centrifuging a whole cell broth to produce a first pellet of cells and protein proximal to the cells (e.g. adherent to the cells, on the surface of the cells) and/or insoluble protein (e.g. protein aggregates) and first supernatant, then discarding the first supernatant to obtain the first pellet. The first pellet was re-suspended in guanidine thiocyanate to solubilize Ds_ACB. The re-suspension was centrifuged again produce a second pellet and a second supernatant. The second supernatant was then dialyzed against PBS and subject to high temperature in order to denature proteins other than Ds_ACB and centrifuged to produce a third pellet and third supernatant. The third supernatant was subject to coacervation by chilling to yield phase separation into a dense lower layer containing Ds_ACB and an upper layer. These samples are referred to in Table 2 below and elsewhere herein as the “gel layer” samples. In some gel layer samples, multiple coacervations were performed by retaining the lower layer and incubating the lower layer at a lower temperature to induce further phase separation. These gel layer samples are referred to in Table 2 below and elsewhere herein as the “first coacervation” and “second coacervation” samples.

A third set of samples was prepared by centrifuging a whole cell broth to produce a pellet and supernatant, then discarding the supernatant to obtain a pellet of cells and protein proximal to the cells (e.g. adherent to the cells, on the surface of the cells) and/or insoluble protein (e.g. protein aggregates). The pellet of cells was re-suspended in guanidine thiocyanate to solubilize the protein that was proximal to the cells. The re-suspension was centrifuged again produce a second pellet of cells and a second supernatant. The second supernatant was then precipitated with ammonium sulfate and centrifuged to produce a third pellet and third supernatant. The third pellet was suspended in guanidine thiocyanate, then dialyzed against PBS and subject to high temperature to denature proteins other than Ds_ACB and centrifuged to produce a fourth supernatant and fourth pellet. The fourth supernatant was then subject to coacervation by chilling to yield phase separation. These samples are referred to in Table 2 below and elsewhere herein as the “gel layer precipitated” samples.

A single sample was produced by adding urea to a whole cell broth to solubilize the protein, then centrifuging the whole cell broth to produce a first pellet and first supernatant. The first supernatant was then precipitated using ammonium sulfate and centrifuged to produce a second pellet and second supernatant. The second supernatant was discarded and the second pellet was then re-suspended in guanidine thiocyanate and dialyzed against PBS, then subject to high temperature in order to denature proteins other than Ds_ACB and centrifuged again to produce a third pellet and a third supernatant. The third supernatant was then coacervated by chilling the third supernatant to induce a phase separation into a dense lower layer containing Ds_ACB and an upper layer. This sample is referred to in Table 2 below and elsewhere herein as the “Urea WCBE” sample.

Another single sample was prepared by centrifuging a whole cell broth to produce a first pellet and first supernatant, then discarding the first supernatant to obtain a first pellet of cells and protein proximal to the cells (e.g. adherent to the cells, on the surface of the cells) and/or insoluble protein (e.g. protein aggregates). The first pellet of cells was re-suspended in guanidine thiocyanate to solubilize the protein. The re-suspension was centrifuged again to produce a second pellet of cells and a second supernatant. The second supernatant was then dialyzed against PBS and then centrifuged to produce a heavy phase of protein, a light phase of supernatant and a film separating the heavy phase from the light phase. The heavy phase of protein was then isolated by discarding the light phase and the film. This sample is referred to in Table 2 below and elsewhere herein as the “Dense layer” sample.

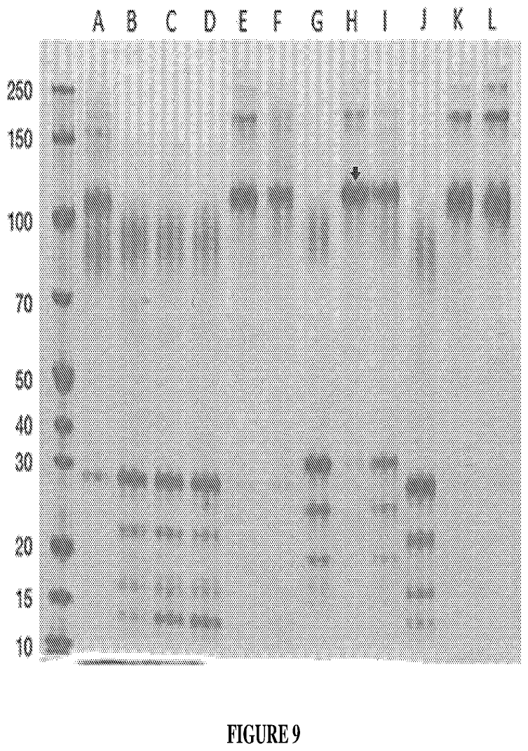

Table 2 (below) lists the various combinations of strains and recovery techniques along with the relative amount of degradation seen in the gel pictured at FIG. 9. As shown in FIG. 9, samples E, F, G, K and L showed bands at approximately 110 kDa and minimal or faint bands at lower molecular weights (labeled in Table 2 as “Minimal”). Samples A, B, C, D, G, I and J had degradation products corresponding to bands at approximately 90, 30, 22, 17 and 12 kDa (labelled in Table 2 as “Substantial”). Among these, samples A and I also showed bands at approximately 110 kDa indicating the presence of full-length resilin. Accordingly, the “Gel layer” samples produced full-length resilin while the CCB samples produced degradation products sometimes in addition to full-length resilin (e.g. sample A) or without full-length resilin (e.g. samples C and D). The Urea WCBE sample only produced degradation products. CCB/gel layer precipitated indicates the combination of isolated material from both the CCB purification and the gel layer purification methods.

TABLE 2
Samples from recovery methods yielding full-length resilin and degradation products

| | | | | | 110 kDa | |
|---|---|---|---|---|---|---|
| Sample | Strain | FLAG | Description | Coacervation | band? | Degradation |
| A | RMs1221 | − | CCB | First | Yes | Substantial |
| B | RMs1221 | − | Urea WCBE | None | No | Substantial |
| C | RMs1221 | − | CCB | First | No | Substantial |
| D | RMs1221 | − | CCB | Second | No | Substantial |
| E | RMs1221 | − | Gel layer | First | Yes | Minimal |
| F | RMs1221 | − | Gel layer | Second | Yes | Minimal |
| G | RMs1209 | + | CCB | First | No | Substantial |
| H | RMs1209 | + | Gel layer | First | Yes | Minimal |
| | | | precipitated | | | |
| I | RMs1209 | + | CCB/gel layer | First | Yes | Substantial |
| | | | precipitated | | | |
| J | RMs1221 | − | CCB | First | No | Substantial |
| K | RMs1221 | − | Gel layer | First | Yes | Minimal |
| L | RMs1221 | − | Dense layer | None | Yes | Minimal |

To verify that the 110 kDa bands shown in samples A, I, E, F, G, K and L corresponded to the full-length resilin (SEQ ID NO: 1), the 110 kDa band in sample H (indicated in FIG. 9 with an arrow) was excised and sent for N-terminus sequencing by Edman degradation. Edman degradation is a cyclic procedure where amino acid residues are cleaved off one at a time and identified by chromatography. There are 3 steps in the cyclic procedure. In step 1 the PITC reagent is coupled to the N-terminal amino group under alkaline conditions. In step 2 the N-terminal residue is cleaved in acidic media. In step 3, the PITC coupled residue is transferred to a flask, converted to a PTH-residue and identified by HPLC chromatography. The next cycle is then started for identification of the next N-terminal residue. Edman degradation analysis was performed on a Shimadazu PPSQ-33 sequencer and a PVDF membrane.

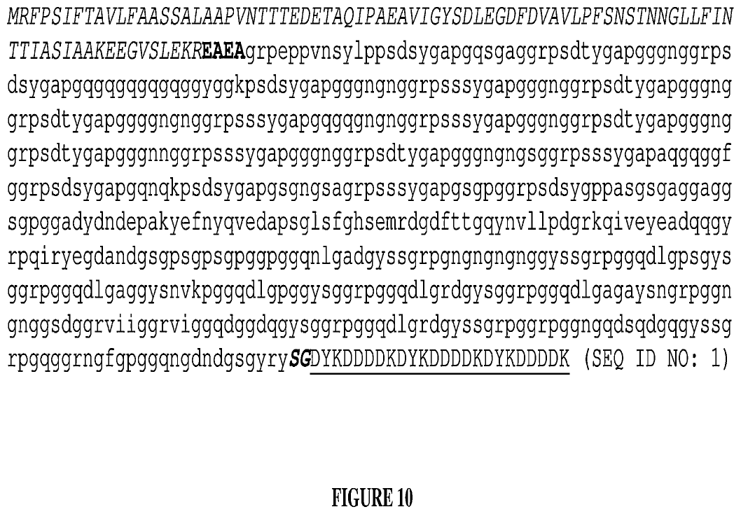

FIG. 10 shows the full-length Drosophila sechellia resilin sequence (Ds_ACB) that is expressed along with signal sequences that are later cleaved. The first sequence (italics), is an alpha mating factor precursor protein signal sequence (SEQ ID NO: 46) that is cleaved twice after transcription by a signal peptidase followed by cleavage with Kex2. The second sequence (bold) is an EAEA repeat that is cleaved by Ste13 (SEQ ID NO: 47). The third sequence (lower case) corresponds to Drosophila sechellia full-length resilin (SEQ ID NO 1). The fourth sequence (bold and italicized) corresponds to a linker sequence (SEQ ID NO: 46). The fifth sequence (underlined) corresponds to the 3X FLAG tag (SEQ ID NO: 45).

Edman sequencing confirmed that the N-terminus of the protein sequences at the approximately 110 kDa band corresponded to the full-length length Drosophila sechellia resilin sequence. Specifically, the N-terminus sequencing showed that the N-terminus either corresponded to “EAEA” or “GRPE”, respectively the full-length Drosophila sechellia resilin sequence with or without the EAEA repeat.

### Example 8: Quantifying the stability of crosslinked resilin

Resilin samples generated by the methods described in Example 7 with varying levels of degradation products and full-length resilin were subject to enzymatic cross-linking as described above with respect to Example 4. The stability of the cross-linked samples was assessed over time by determining the duration each cross-linked samples remained a solid through daily observation. Table 3 shows the time as a solid for each cross-linked sample. As shown in Table 3, samples comprising full-length resilin had a longer duration of stability than the samples that did not comprise full-length resilin.

TABLE 3
Stability of cross-linked resilin

| Sample | 110 kDa band? | Degradation | Time as solid |
|---|---|---|---|
| A | Yes | Substantial | 13 days |
| B | No | Substantial | 8 days |
| C | No | Substantial | 6 days |
| D | No | Substantial | 6 days |
| E | Yes | Minimal | N/A |
| F | Yes | Minimal | 27 days |
| G | No | Substantial | 8 days |
| H | Yes | Minimal | 15 days |
| I | Yes | Substantial | 15 days |
| J | No | Substantial | 6 days |
| K | Yes | Minimal | 13 days |
| L | Yes | Minimal | 13 days |

### ADDITIONAL CONSIDERATIONS

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the claims.

### SEQUENCE LISTING

| SEQ ID | | |
|---|---|---|
| NO: | Species | Sequence |
| 1 | Drosophila | RPEPPVNSYLPPSDSYGAPGQSGAGGRPSDTYGAPGGGNGGRPSDSYGAPG |
| | sechellia | QGQGQGQGQGGYGGKPSDSYGAPGGGNGNGGRPSSSYGAPGGGNGGRPSDT |
| | | YGAPGGGNGGRPSDTYGAPGGGGNGNGGRPSSSYGAPGQGQGNGNGGRPSS |
| | | SYGAPGGGNGGRPSDTYGAPGGGNGGRPSDTYGAPGGGNNGGRPSSSYGAP |
| | | GGGNGGRPSDTYGAPGGGNGNGSGGRPSSSYGAPAQGQGGFGGRPSDSYGA |
| | | PGQNQKPSDSYGAPGSGNGSAGRPSSSYGAPGSGPGGRPSDSYGPPASGSG |
| | | AGGAGGSGPGGADYDNDEPAKYEFNYQVEDAPSGLSFGHSEMRDGDFTTGQ |
| | | YNVLLPDGRKQIVEYEADQQGYRPQIRYEGDANDGSGPSGPSGPGGPGGQN |
| | | LGADGYSSGRPGNGNGNGNGGYSSGRPGGQDLGPSGYSGGRPGGQDLGAGG |
| | | YSNVKPGGQDLGPGGYSGGRPGGQDLGRDGYSGGRPGGQDLGAGAYSNGRP |
| | | GGNGNGGSDGGRVIIGGRVIGGQDGGDQGYSGGRPGGQDLGRDGYSSGRPG |
| | | GRPGGNGQDSQDGQGYSSGRPGQGGRNGFGPGGQNGDNDGSGYRY |
| 2 | Drosophila | RPEPPVNSYLPPSDSYGAPGQSGAGGRPSDTYGAPGGGNGGRPSDSYGAPG |
| | sechellia | QGQGQGQGQGGYGGKPSDSYGAPGGGNGNGGRPSSSYGAPGGGNGGRPSDT |
| | | YGAPGGGNGGRPSDTYGAPGGGGNGNGGRPSSSYGAPGQGQGNGNGGRPSS |
| | | SYGAPGGGNGGRPSDTYGAPGGGNGGRPSDTYGAPGGGNNGGRPSSSYGAP |
| | | GGGNGGRPSDTYGAPGGGNGNGSGGRPSSSYGAPAQGQGGFGGRPSDSYGA |
| | | PGQNQKPSDSYGAPGSGNGSAGRPSSSYGAPGSGPGGRPSDSYGPPASG |
| 3 | Drosophila | GKPSDSYGAPGGGNGNGGRPSSSYGAPGGGNGGRPSDTYGAPGGGNGGRPS |
| | sechellia | DTYGAPGGGGNGNGGRPSSSYGAPGQGQGNGNGGRPSSSYGAPGGGNGGRP |
| | | SDTYGAPGGGNGGRPSDTYGAPGGGNNGGRPSSSYGAPGGGNGGRPSDTYG |
| | | APGGGNGNGSGGRPSSSYGAPAQGQGGFGGRPSDSYGAPGQNQKPSDSYGA |
| | | PGSGNGS |
| 4 | Acromyrmex | FGENRGNGGKPSTSYGVPDSNGNNRGGFGNGGSEGRPSTSYGLPDASRNNG |
| | echinatior | NGFGNVGNEDKPSTNYGIPANGNKVSGFGNVGSEGRPSTSYGVPGANGNQG |
| | | FGSGGIGGRPSTSYGVPGVNGNNGGGFENVGRPSTSYGTPDARGNNGGSFR |
| | | NGDIGGRPSTNYGIPGANGNHG |
| 5 | Aeshna | APSRGGGHGGGSISSSYGAPSKGSGGFGGGSISSSYGAPSKGSVGGGVSSS |
| | | YGAPAIGGGSFGGGSFGGGSFGGGSFGGGAPSSSYGAPSSSYSAPSSSYGA |
| | | PSKGSGGFGSSGGFSSFSSAPSSSYGAPSASYSTPSSSYGAPSSGGFGAGG |
| | | GFSSG |
| 6 | Aeshna | EPPVGGSQSYLPPSSSYGAPSAGTGFGHGGGSPSQSYGAPSFGGGSVGGGS |
| | | HFGGGSHSGGGGGGYPSQSYGAPSRPSGSSFQAFGGAPSSSYGAPSSQYGA |
| | | PSGGGGSYAIQGGSFSSGGSRAPSQAYGAPSNNAGLSHQSQSFGGGLSSSY |
| | | GAPSAGFGGQSHGGGYSQGGNGGGHGGSSGGGYSYQSFGGGNGGGHGGSRP |
| | | SSSYGAPSSSYGAPSGGKGVSGGFVSQPSGSYGAPSQSYGAPSRGGGHGGG |
| | | SISSSYGAPSKGSGGFGGGSISSSYGAPSKGSVGGGVSSSYGAPAIGGGSF |
| | | GGGSFGGGSFGGGSFGGGAPSSSYGAPSSSYSAPSSSYGAPSKGSGGFGSS |
| | | GGFSSFSSAPSSSYGAPSASYSTPSSSYGAPSSGGFGAGGGFSSGGYSGGG |
| | | GGYSSGGSGGFGGHGGSGGAGGYSGGGGYSGGGSGGGQKYDSNGGYVYS |
| 7 | Haematobia | AGGGNGGGGTGGTPSSSYGAPSNGGGSNGNGFGSPSSSYGAPGSGGSNGNG |
| | irritans | GGRPSLSYGAPGSGGSNGNGGGRPSSSYGAPGAGGSNGNGGGRPSSSYGAP |
| | | GAGGSNGNGGGRPSSSYGAPGAGGSNGNGGGRPSSSYGAPGAGGSNGNGGS |
| | | RPSSTYGAP |
| 8 | Haematobia | RPEPPVNSYLPPPLNNYGAPGAGGGSSDGSPLAPSDAYGAPDLGGGSGGSG |
| | irritans | QGPSSSYGAPGLGGGNGGAPSSSYGAPGLGGGNGGSRRPSSSYGAPGAGGG |
| | | NGGGGTGGTPSSSYGAPSNGGGSNGNGFGSPSSSYGAPGSGGSNGNGGGRP |
| | | SLSYGAPGSGGSNGNGGGRPSSSYGAPGAGGSNGNGGGRPSSSYGAPGAGG |
| | | SNGNGGGRPSSSYGAPGAGGSNGNGGGRPSSSYGAPGAGGSNGNGGSRPSS |
| | | TYGAPGAGGSNGNGCGNKPSSSYGAPSAGSNGNGGSEQGSSGSPSDSYGPP |
| | | ASGTGRGRNGGGGGAGGGRRGQPNQEYLPPNQGDNGNNGGSGGDDGYDYSQ |
| | | SGDGGGQGGSGGSGNGGDDGSNIVEYEAGQEGYRPQIRYEGEANEGGQGSG |
| | | GAGGSDGTDGYEYEQNGGDGGAGGSGGPGTGQDLGENGYSSGRPGGDNGGG |
| | | GGYSNGNGQGDGGQDLGSNGYSSGAPNGQNGGRRNGGGQNNNGQGYSSGRP |
| | | NGNGSGGRNGNGGRGNGGGYRNGNGNGGGNGNGSGSGSGNNGYNYDQQGSN |
| | | GFGAGGQNGENDGSGYRYS |
| 9 | Ctenocephalides | ANGNGFEGASNGLSATYGAPNGGGFGGNGNGGAPSSSYGAPGAGNGGNGGG |
| | felis | RPSSSYGAPGAGGSGNGFGGRPSSSYGAPGNGNGANGGRGGRPSSRYGAPG |
| | | NGNGNGNGNGGRPSSSYGAPGSNGNGGRPSSSYGAPGSGNGFGGNGGRPSS |
| | | SYGAPGANGNGNGGAIGQPSSSYGAPGQNGNGGGLSSTYGAPGAGNGGFGG |
| | | NGGGLSSTYGAPGSGNGGFGGNGLSSTYGAPGSGNGGFGGNGGGLSSTYGA |
| | | P |
| 10 | Ctenocephalides | PGGAGGAGGYPGGAGGAGGAGGYPGGSAGGAGGYPGGSGSGVGGYPGGSNG |
| | felis | GAGGYPGGSNGGAGGYPGGSNGGAGGYPGGSNGGAGGYPGGSNGNGGYSNG |
| | | GSNGGGAGGYPGGSNGNGGYPGSGSNGGAGGYPGGSNGNGGYPG |
| 11 | Bombus | FDGQNGIGGGDSGRNGLSNSYGVPGSNGGRNGNGRGNGFGGGQPSSSYGAP |
| | terrestris | SNGLGGNGGSGAGRPSSSYGAPGGGNGFGGGQPSSSYGAPSNGLGGNGAGR |
| | | PSSSYGAPGGGNGFGGGSNGAGKNGFGGAPSNSYGPPENGNGFGGGNGGGS |
| | | PSGLYGPPGRNGGNGGNGGNGGNGGRPSSSYGTPERNGGRPSGLYGPP |
| 12 | Tribolium | NGFGGGQNGGRLSSTYGPPGQGGNGFGGGQNGGRPSSTYGPPGQGGNGFGG |
| | castaneum | GQNGGRPSSTYGPPGQGGNGFGGGQNGGRPSSTYGPPGQGGNGFGGGQNGG |
| | | RPSSTYGPPGQGGNGFGGGQNGGRPSSTYGPPGQGGNGFGGGQNGGKPSST |
| | | YGPPGQGGNGFGGGQNGGRPSSTYGPPGQG |
| 13 | Tribolium | RAEPPVNSYLPPSQNGGPSSTYGPPGFQPGTPLGGGGNGGHPPSQGGNGGF |
| | castaneum | GGRHPDSDQRPGTSYLPPGQNGGAGRPGVTYGPPGQGGGQNGGGPSSTYGP |
| | | PGQGGNGFGGGQNGGRLSSTYGPPGQGGNGFGGGQNGGRPSSTYGPPGQGG |
| | | NGFGGGQNGGRPSSTYGPPGQGGNGFGGGQNGGRPSSTYGPPGQGGNGFGG |
| | | GQNGGRPSSTYGPPGQGGNGFGGGQNGGRPSSTYGPPGQGGNGFGGGQNGG |
| | | KPSSTYGPPGQGGNGFGGGQNGGRPSSTYGPPGQGGNGNGGGHNGQRPGGS |
| | | YLPPSQGGNGGYPSGGPGGYPSGGPGGNGGYGGEEESTEPAKYEFEYQVDD |
| | | DEHNTHFGHQESRDGDKATGEYNVLLPDGRKQVVQYEADSEGYKPKISYEG |
| | | GNGNGGYPSGGPGGAGNGGYPSGGPQGGNGGYPSGGPQGGNGGYPSGGPQG |
| | | GNGGYPSGGPQGGNGGYPSGGPQGGNGGYPSGGPQGGNGGYPSGGPQGGNG |
| | | GYPSGGPQGGNGGYTSGGPQGGNGGYPSGGPQGGNGGSGPY |
| 14 | Tribolium | QLTKRDAPLSGGYPSGGPANSYLPPGGASQPSGNYGAPSGGFGGKSGGFGG |
| | castaneum | SGGFGGAPSQSYGAPSGGFGGSSSFGKSGGFGGAPSQSYGAPSGGFGGSSS |
| | | FGKSSGGFGGAPSQSYGAPSGGFGGSSSFGKSGGFGGAPSQSYGAPSGGFG |
| | | GSSSFGKSGGFGGAPSQSYGAPSGGFGGKSSSFSSAPSQSYGAPSGGFGGK |
| | | SGGFGGAPSQSYGAPSGGFGGKSGGFGGAPSQSYGAPSGGFGGSSSFGKSG |
| | | GFGGAPSQSYGAPSGGFGGSSSFGKSSGFGHGSGAPSQSYGAPSRSQPQSN |
| | | YLPPSTSYGTPVSSAKSSGSFGGAPSQSYGAPSQSHAPSQSYGAPSRSFSQ |
| | | APSQSYGAPSQGHAPAPQQSYSAPSQSYGAPSGGFGGGHGGFGGQGQGFGG |
| | | GRSQPSQSYGAPAPSQSYGAPSAGGQQYASNGGYSY |
| 15 | Apis mellifera | RSEPPVNSYLPPSGNGNGGGGGGSSNVYGPPGFDGQNGIGEGDNGRNGISN |
| | | SYGVPTGGNGYNGDSSGNGRPGTNGGRNGNGNGRGNGYGGGQPSNSYGPPS |
| | | NGHGGNGAGRPSSSYGAPGGGNGFAGGSNGKNGFGGGPSSSYGPPENGNGF |
| | | NGGNGGPSGLYGPPGRNGGNGGNGGNGGRPSGSYGTPERNGGRLGGLYGAP |
| | | GRNGNNGGNGYPSGGLNGGNGGYPSGGPGNGGANGGYPSGGSNGDNGGYPS |
| | | GGPNGNGNGNGGYGQDENNEPAKYEFSYEVKDEQSGADYGHTESRDGDRAQ |
| | | GEFNVLLPDGRKQIVEYEADQDGFKPQIRYEGEANSQGYGSGGPGGNGGDN |
| | | GYPSGGPGGNGYSSGRPNGGSDFSDGGYPSTRPGGENGGYRNGNNGGNGNG |
| | | GYPSGNGGDAAANGGYQY |
| 16 | Apis mellifera | DAPISGSYLPPSTSYGTPNLGGGGPSSTYGAPSGGGGGRPSSSYGAPSSTY |
| | | GAPSSTYGAPSNGGGRPSSTYGAPSNGGGRPSSSYGAPSSSYGAPSSTYGA |
| | | PSNGGGRPSSSYGAPSFGGGGGFGGGNGLSTSYGAPSRGGGGGGGSISSSY |
| | | GAPTGGGGGGPSTTYGAPNGGGNGYSRPSSTYGTPSTGGGSFGGSGGYSGG |
| | | GGGYSGGGNGYSGGGGGGYSGGNGGGYSGGGNGGGYSGGNGGGYSGGGGGG |
| | | YSGGGGGGYSGGGNGYSGGGGGGYSGGNGGYSGGNGGYSGGGGGYSGGGGG |
| | | GQSYASNGGYQY |
| 17 | Nasonia | RPEPPVNSYLPPGQGGGFGGGRPSGASPSDQYGPPDFQGAGGRGGQAAGGN |
| | vitripennis | FGGGGNGFGGAPSSSYGPPGFGSNEPNKFSGAGGGGAGRPQDSYGPPAGGN |
| | | GFAGSAGAGNSGRPGGAAAGGRPSDSYGPPQGGGSGFGGGNAGRPSDSYGP |
| | | PSAGGGGFGGGSPGGGFGGGSPGGGFGGGNQGAPQSSYGPPASGFGGQGGA |
| | | GQGRPSDSYGPPGGGSGGRPSQGGNGFGGGNAGRPSDSYGPPAAGGGGFGG |
| | | NAGGNGGGNGFGGGRPSGSPGGFGGQGGGGRPSDSYLPPSGGSGFGGGNGR |
| | | QPGGFGQQGGNGAGQQNGGGGAGRPSSSYGPPSNGNGGGFSGQNGGRGSPS |
| | | SGGGFGGAGGSPSSSYGPPAGGSGFGNNGGAGGRPSSSYGPPSSGGNGFGS |
| | | GGQGGQGGQGGQGGRPSSSYGPPSNGNGGFGGGNGGRPSSNGYPQGQGNGN |
| | | GGFGGQGGNGGRPSSSYGPPGGDSGYPSGGPSGNFGGSNAGGGGGGFGGQV |
| | | QDSYGPPPSGAVNGNGNGYSSGGPGGNGLDEGNDEPAKYEFSYEVKDDQSD |
| | | GRKQIVEYEADQDGFKPQIRYEGEANTGAGGAGGYPSGGGGDSGYPSGPSG |
| | | AGGNAGYPSGGGGGAGGFGGNGGGSNGYPSGGPSGGQGQFGGQQGGNGGYP |
| | | SGPQGGSGFGGGSQGSGSGGYPSGGPGGNGGNNNFGGGNAGYPSGGPSGGN |
| | | GFNQGGQNQGGSGGGYPSGSGGDAAANGGYQYS |
| 18 | Nasonia | RAEAPISGNYLPPSTSYGTPNLGGGGGGGGGFGGGAPSSSYGAPSSGGGFG |
| | vitripennis | GSFGGGAPSSSYGAPSTGGSFGGGAPSSSYGAPSSGGSFGGSFGGGAPSSS |
| | | YGAPSFGGNAPSSSYGAPSAGGSFGGGAPSNSYGPPSSSYGAPSAGGSFGG |
| | | SSGGSFGGSFGGGAPSSSYGAPAPSRPSSNYGAPSRPSSNYGAPSSGGSGF |
| | | GGGSGFGGGRPSSSYGAPSSGSFGGGFGGGAPSSSYGAPAPSRPSSNYGAP |
| | | APSRPSSNYGAPAPSRPSSSYGAPSRPSSNYGAPSRPSSNYGAPSSGGSGF |
| | | GGGSGFGGGRPSSSYGAPSSGSFGGGFGGGAPSSSYGAPAPSRPSSNYGPP |
| | | SSSYGAPSSGGSGGFGGGAPSSSYGAPSFGGSSNAVSRPSSSYGAPSSGGG |
| | | QSYASNGGYQY |
| 19 | Pediculus | EPPVKTSYLPPSASRSLNSQYGAPAFTDSNELVAPSPNSNFHDSYNQQQQS |
| | humanus | FDLSNGLSVPSAAGRLSNTYGVPSAQGANVPSFDSSDSIAVDAAGRSGNSF |
| | corporis | SSHVPSSTYGAPGNGFGGGSRSSQSGAPSSVYGPPQARNNNFGNGAAPSSV |
| | | YGPPQARNNNFGNGGAPSQVYGPPKARNNNFGNGAAPSSVYGPPQARNNNF |
| | | GNGAAPSSVYGPPQARNNNFANSAAPSQVYGPPQARNNNFGNGAAPSSVYG |
| | | PPQSSSFSSPSGRSGQLPSATYGAPFERNGFGSQGSSGFQGYEPSKRSQTT |
| | | EDPFAEPAKYEYDYKVQASDETGTEFGHKESRENESARGAYHVLLPDGRMQ |
| | | IVQYEADETGYRPQIRYEDTGYPSAASSRSNNGFNGYQY |
| 20 | Anopheles | KREAPLPPSGSYLPPSGGAGGYPAAQTPSSSYGAPTGGAGSWGGNGGNGGR |
| | gambiae str. | GHSNGGGSSFGGSAPSAPSQSYGAPSFGGQSSGGFGGHSSGGFGGHSSGGH |
| | PEST | GGNGNGNGNGYSSGRPSSQYGPPQQQQQQQSFRPPSTSYGVPAAPSQSYGA |
| | | PAQQHSNGGNGGYSSGRPSTQYGAPAQSNGNGFGNGRPSSSYGAPARPSTQ |
| | | YGAPSAGNGNGYAGNGNGRSYSNGNGNGHGNGHSNGNGNNGYSRGPARQPS |
| | | QQYGPPAQAPSSQYGAPAQTPSSQYGAPAQTPSSQYGAPAQTPSSQYGAPA |
| | | QTPSSQYGAPAPSRPSQQYGAPAPSRPSQQYGAPAQTPSSQYGAPAQTPSS |
| | | QYGAPAQTPSSQYGAPAQTPSSQYGAPAQQPSSQYGAPAPSRPSQQYGAPA |
| | | QQPSAQYGAPAQTPSSQYGAPAPSRPSQQYGAPAQAPSSQYGAPAPSSQYG |
| | | APAQQPSSQYGAPAQTPSSQYGAPSFGPTGGASFSSGNGNVGGSYQVSSTG |
| | | NGFSQASFSASSFSPNGRTSLSAGGFSSGAPSAQSAGGYSSGGPSQVPATL |
| | | PQSYSSNGGYNY |
| 21 | Glossina | RPEPPVNTYLPPSAGGGSGGGSPLAPSDTYGAPGVNGGGGGGGGPSSTYGA |
| | morsitans | PGSGGGNGNGGGGFGKPSSTYGAPGLGGGGNGGGRPSETYGAPSGGGGNGF |
| | | GKPSSTYGAPNGGGGNGGPGRPSSTYGAPGSGGGNGGSGRPSSTYGAPGLG |
| | | GGNGGSGRPSSMYGAPGLGGGNGGSGRPSSTYGAPGSGGGNGGSGRPSSTY |
| | | GAPGSGGGNGGSGRPSSTYGAPGNGNGGNGFGRPSSTYGAPGSGGSNGNGK |
| | | PSSTYGAPGSGGGGGRPSDSYGPPASGNGGRNGNGNGQSQEYLPPGQSGSG |
| | | GGGGYGGGSGSGGSGGGGGGGYGGDQDNNVVEYEADQEGYRPQIRYEGDGS |
| | | QGGFGGDGDGYSYEQNGVGGDGGGAGGAGGYSNGQNLGANGYSSGRPNGGN |
| | | GGGRRGGGGGGGGSGGGQNLGSNGYSSGAPNGFGGGNGQGYSGGRSNGNGG |
| | | GGGGRNGGRYRNGGGGGGGRNGGGSNGYNYDQPGSNGFGRGGGNGENDGSG |
| | | YHY |
| 22 | Atta cephalotes | RSEPPVNSYLHPGSDTSGTNGGRTDLSTQYGAPDFNNRGNGNSGATSFGGS |
| | | GAGNGPSKLYDVPIRGNTGGNGLGQFRGNGFESGQPSSSYGAPKGGFGENR |
| | | GNRGRPSTSYGVPDSNRNNRGGFGNGGSEARPSTSYGVPGANGNQGGFGSG |
| | | SIGGRPSTSYGVPGANGNNGDSFRNGDIGGRPSTNYGAPGANGNHGGGNGG |
| | | NGRPSNNYGVPGANGNTNGKGRLNGNSGGGPSNNYGSPNGFGKGLSTSYGS |
| | | PNRGGNDNHYPSRGSFINGGINGYSSGSPNGNAGNFGHGDESFGRGGGEGE |
| | | NTGEGYNANAQEESTEPAKYEFSYKVKDQQTGSDYSHTETRDGDHAQGEFN |
| | | VLLPDGRKQIVEYEADQDGFKPQIRYEGEANADGGYGSGLNDNNDGYSSGR |
| | | PDSESGGFANSGFNGGSSNGGYPNGGPGERKLGGFNNGGSSGYQSGRSAGQ |
| | | SFGRDNAGDLNNDIGGYFSNSPNNIGDSDNANVGSNRQNDGNSGYQY |
| 23 | Anopheles | KREAPLPPSGSYLPPSGGGGGGGGYPAAQTPSSSYGAPAGGAGGWGGNGNG |
| | darlingi | NGNGNGGRGGYSNGGGHSGSAPSQSYGAPSAPSQSYGAPSQSYGAPAAAPS |
| | | QSYGAPSFGGNGGGASHGSGGFTGGHGGNGNGNGYSSGRPSSQYGPPQQQQ |
| | | QPQQQSFRPPSTSYGVPAAPSSSYGAPSANGFSNGGRPSSQYGAPAPQSNG |
| | | NEFGAPRPSSSYGAPSRPSTQYGAPSNGNGNGYAGHGNGNGHGNGNGHSNG |
| | | NGNGYNRGPARQPSSQYGPPSQGPPSSQYGPPSQYGPPSSGTSFIAYGPPS |
| | | QGPPSSQYGAPAPSRPSSQYGAPAQTPSSQYGAPAQTPSSQYGPPRQSSPQ |
| | | FGAPAPRPPSSQYGAPAQAPSSQYGAPAQTPSSQYGAPAQAPSSQYGAPAP |
| | | SRPSSQYGVPAQAPSSQYGAPAQAPSSQYGAPAQTPSSQYGAPSFGSTGGS |
| | | SFGGNGGVGGSYQTASSGNGFSQASFSASSFSSNGRSSQSAGGYSSGGPSQ |
| | | VPATIPQQYSSGGGSYSSGGHSQVPATLPQQYSSNGGYNY |
| 24 | Acromyrmex | RSEPPVNSYLPPGPGTSGANGGQTDLSIQYRASDFNNRGNVNGNSGATSFG |
| | echinatior | GPGASNGPSKLYDVPIGGNAGGNGLGQFRGNGFEGGQPSSSYGAPNGGFGE |
| | | NRGNGGKPSTSYGVPDSNGNNRGGFGNGGSEGRPSTSYGLPDASRNNGNGF |
| | | GNVGNEDKPSTNYGIPANGNKVSGFGNVGSEGRPSTSYGVPGANGNQGFGS |
| | | GGIGGRPSTSYGVPGVNGNNGGGFENVGRPSTSYGTPDARGNNGGSFRNGD |
| | | IGGRPSTNYGIPGANGNHGGGNGGNGRPSSNYGVPGGNGNTNGKGRFNGNS |
| | | GGRPSNSYGSPNGFGKGLSTSYSPSNRDGNGNHYPSGDSNRGSFVNGGING |
| | | YPSGSPNGNAGNFRHGDESFGRGGEGGGRSTGEGYNANAQEESTEPAKYEF |
| | | SYKVKDQQTGSDYSHTETRDGDHAQGEFNVLLPDGRKQIVEYEADQDGFKP |
| | | QIRYEGEANADGEYDSGGLNDNNDGYSSGRPGSESGGFANNSGFNGGSSNG |
| | | GYPSGGSGEGKLGFNSGGNSGYQSGRPAGQSFGRDNAGDLSNDIGGFSNSP |
| | | NNIGGDNANVGSNRQNGGNSGYQY |
| 25 | Acyrthosiphon | ESPYGGGSSNSNGNGRNGGYGGKGQYGGGNGGGVGSSSASPFFSGANQYGS |
| | pisum | QSGLSGAANNRYPSFGSKFGGNKGSYGGSSSRNNGRYGSGSASGYGSGSSG |
| | | GLGSTGRSTGGYGGGSSGSYGSGSSGSLGSSTGSNGIYGAGSSGGFGSGSS |
| | | GSYGGGSSGGFGSGSSGSYGGGSSGGFGSGSSGSYGGGSSGGFGSGSSGSY |
| | | GGGSSGGFGSGSSGNYGSGSSGSYGSGGGGLGGASSGNNDGYGAGGSGSYD |
| | | QLGGANGNGLGGSGNDPLSEPANYEFSYEVNAPESGAIFGHKESRQGEEAT |
| | | GVYHVLLPDGRTQIVEYEADEDGYKPKITYTDPVGGYAGDRQSGNSYGGNG |
| | | GFGGSGSLGGSGGNLGGLYNGGGSSNNGAGYGGSSSSLGSRYGGSGGSSGS |
| | | GVGGGYGGSGSSSGGIGSSYGGSGSLSGGLGGGYGGSGSSSGGLGGGYGGS |
| | | GGSSGGGFGGLGGSGGSSGSGYGGSGSSSGGLGNSYGGSGSSNGGLGGGYS |
| | | GSGGSSGGLGGGYGASSGSSGSGLGGGYGGSGSSSGGLGSGYGGLGSSSGG |
| | | LGGGYGGSGSSSGGLGGGYGGSGSSNGGIGGGYGGSSGSSGGLGGGYGGSG |
| | | SSSGGLGGGYGGSGGSNSGLGSSYGGSGSTNGGLGGGYGGLGSSSGGLGGG |
| | | YGGSGGSNGGIGGGYGGSSGSGGSQGSAYGGSGSSSGSQGGGYGGSGSSSG |
| | | GLGGGYGSSSGSSSGLGGSYGSNRNGLGSGSSYS |
| 26 | Drosophila | RPEPPVNSYLPPSPGDSYGAPGQGQGQGQGGFGGKPSDSYGAPGAGNGNGN |
| | virilis | GRPSSSYGAPGQGQGQGGFGGKPSDSYGAPGAGNGNGNGRPSSSYGAPGQG |
| | | QGQGGFGGRPSSSYGAPGQGQGGFGGKPSDTYGAPGAGNGNGRPSSSYGAP |
| | | GQGQGGIGGKPSDSYGAPGAGNGNGNGRPSSSYGAPGQGQGGFGGKPSDTY |
| | | GAPGAGNGNGRPSSSYGAPGQGQGGFGGKPSDTYGAPGAGNGNGNGRPSSS |
| | | YGAPGQGQGGFGGKPSDTYGAPGAGNGNGRPSSSYGAPGQGQGQGGFGGKP |
| | | SDSYGPPASGAGAGGAGGPGAGGGGDYDNDEPAKYEFNYQVEDAPSGLSFG |
| | | HSEMRDGDFTTGQYNVLLPDGRKQIVEYEADQQGYRPQVRYEGDANGNGGP |
| | | GGAGGPGGQDLGQNGYSSGRPGGQDLGQGGYSNGRPGGQDLGQNGYSGGRP |
| | | GGQDLGQNGYSGGRPGGQDLGQNGYSGGRPGGQDLGQNGYSGGRPGGQDLG |
| | | QNGYSGGRPGGQDLGQNGYSGGRPGGNGGSDGGRVIIGGRVIGQDGGDGQG |
| | | YSSGRPNGQDGGFGQDNTDGRGYSSGKPGQGRNGNGNSFGPGGQNGDNDGS |
| | | GYRY |
| 27 | Drosophila | RPEPPVNSYLPPSDSYGAPGQSGPGGRPSDSYGAPGGGNGGRPSDSYGAPG |
| | erecta | LGQGQGQGQGQGGFGGKPSDSYGAPGAGNGNGGRPSSSYGAPGAGNGGRPS |
| | | DTYGAPGGGSGGRPSDTYGAPGGGNGNGNGGRPSSSYGAPGQGQGNGNSGR |
| | | PSSSYGAPGAGNGGRPSDTYGAPGGGNGGRPSSSYGAPGAGNGGNGGRPSD |
| | | TYGAPGGGNGNGNGNGNGSGGRPSSSYGAPGQGQGGFGGRPSDSYGAPGQN |
| | | QKPSDSYGAPGSGSGSGNGNGGRPSSSYGAPGSGPGGRPSDSYGPPASGSG |
| | | AGGAGGSGPGGADYDNDIVEYEADQQGYRPQIRYEGDANDGSGPSGPGGQN |
| | | LGADGYSSGRPGNGNGNGNGGYSGGRPGGQDLGPSGYSGGRPGGQDLGAGG |
| | | YSNGRPGGQDLGPSGYSGGRPGGQDLGPSGYSGGRPGGQDLGAGGYSNGRP |
| | | GGNGNGNGGADGGRVIIGGRVIGGQDGGDQGYSGGRPGGQDLGRDGYSSGR |
| | | PGGRPGANGQDNQDGQGYSSGRSGKGGRNSFGPGGQNGDNDGSGYRY |
| 28 | Lutzomyia | RPEPPANTYLPPSSSYAAPGQQGGSGFGGGGGSGGSGGFGQPGAFGRPSSS |
| | longipalpis | YGPPSQGGAGGGFGSDSQFGGGFGGGAGGFGSGGSGAPGASQRPSSSYGPP |
| | | GQTGGGGFGAQGAPGSSFGPGGGFGGGSPGQAGSPGFQRPSSSYGPPGQSP |
| | | GGGFSQQGGAPGASQRPSSTYGAPGQGAGGFGQGGSGGFGGTGGSVAIGGR |
| | | PSSSYGAPGQGSSGGFGGGSGGFGSQAPSTSYGAPGQGSPGGGFGSQGGPG |
| | | GQPGSPGFGGSQRPSSSYGPPGQGGAPGQGGSPGFGASSRSGGAGGFGASQ |
| | | QPSSSYGPPGQGAGSGFQGTGGGFGGPGQRPGFGGSQTPATSYGAPGQAGG |
| | | ASGGFGGAGAQRPSSSYGPPGQASGFGGGSSGGGFGGGSSGGFGGNQGGFG |
| | | GNQGGFGGSQTPSSSYGAPSFGSGGSPGAAGGAGGFGQGGVGGSGQPGGFG |
| | | GGDQGYPPRGGPGGFGPGSGGSGAGGPIAGGSGSGYPGGSDSGSNEPAKYD |
| | | FSYQVDDPASGTSFGHSEQRDGDYTSGQYNVLLPDGRKQIVEYEADLGGYR |
| | | PQIKYEGGSSGGAGGYPSGGPGSQGGAGGYPSGGPGGPGSPGGAGGYQSGA |
| | | AGGAGGYPSGGPGGPGAGGYPSGGPGGPGSQAGGFSGGFGGGSDGAFGGAG |
| | | GFSQGGAGGGDAGYPRGGPGGFGGAGSPGFGGSGSPGFGGSGSPGAQGSSG |
| | | FGGTGGGFGGGADGYPRGGPGAGQSGFQDGRGATGGAGQPGGRGSFGRPGS |
| | | ARGGSSSNGYANGGAEGYPRDNPQNRGSGYS |
| 29 | Rhodnius prolixus | KRDDPLRRFLAPLVGGGNGSGGGGGGYNYNKPANGLSLPGGGGALPPATSY |
| | | GVPDRPAPVPSSPPSSSYGAPQPSPNYGAPSSSYGAPSQQPSRSYGAPSQG |
| | | PSTSYSQRPSSSYGAPAPQTPSSSYGAPAQQPSGSYGAPSGGGGSSGYTGG |
| | | AQRPSGSYGAPSQGGPSGNYGPPSQQPSSNYGAPSQTPSSNYGAPAQRPST |
| | | SYGAPSQPPSSSYGSPPQRASGYPSSSSGPSNGYSPPAQRPSSSYGPPSQQ |
| | | PASSYGAPSQTPSSNYGPPAPIPSSNYGAPSQPPSKPSAPSSSYGTPSQTP |
| | | STSYGAPSQAPSSSYGAPSRPSPPSSSYGAPSQGPSSSYGPPSRPSQPSSP |
| | | SSGYGAPSQGPSSSYGAPSRPSSPSSSYGAPPSSSYGAPSRPSPPSSSYGA |
| | | PSQGPSSSYGPPSRPSQPSSPSSGYGAPSQGPSSSYGAPSRPSSPSSSYGA |
| | | PPSSSYGAPSRPSPPSSSYGAPSQGPSSSYGPPSRPSQPSSPSSSYGAPSQ |
| | | GPSSSYGAPSRPSPPSSSYGAPSQGPSSSYGPPSRPSQPSSTYGVPSGGRP |
| | | STPSSSYGAPPQALSSTYGAPSGRPGAPSQKPSSSYGAPSLGGNASRGPKS |
| | | SPPSSSYGAPSVGTSVSSYAPSQGGAGGFQSSRPSSSYGAPSTGPSSTYGP |
| | | PSQPPSSSYGVPSQPPSSNYGVPSQGVSGSVGSSSPSSSYGAPSQIPSSSY |
| | | GAPSQSSIGGFGSSRPSSSYGAPPQAPSSSYSAPLRAPSTSYGAPSGGSGS |
| | | NFGSKPSTNYGAPSQPPSTNYGPPSQPPSSSYGTPSRAPSPTYSTPQSSGT |
| | | SFGSRPSSSYGVPSQPTTNYGAPSQTPSSNYGAPPASSAPSSTYGRPSQSP |
| | | SSSYGAPSPSSSSSSYESPSQPPSSSYGAPSQGPSSSYGAPSRPSSTYGAP |
| | | SPSSPSTNYGAPAPSSNYGTPAQDLTGSYAAPSQPPSAGYGAPSGQPSSGG |
| | | KQNFQVKNPFAGQTHQVYPAVSSISFGLPSQSFNTAIQGQEPSQSYGAPTA |
| | | SSPSSSYGAPTGTGSSQPGQSYASNGGYSYS |
| 30 | Rhodnius prolixus | QPPFNHYLPAARGSGSNSAQYTAPSSKFGTSTGQYGQPPSEVPRGLQQGSY |
| | | AEDVHSSRSVNPSSQNGIPSGHFSSLSSNYGAPSSDYSRSFLRYGTLSNKY |
| | | GVPNSALGSLSSRNNKTPATQLSYQPSSHYDSRSTSEDQFISSRVSDSQYG |
| | | ASSVRRFLPSSQYSTPSSQYGTPSSQYGTPSSQYGTPSSQYGTPSSQYGTP |
| | | SSQYGTPSSQYGTPSSQYGTPSSQYGTPSSQYGTPSSPPSQYGGPYSMRTS |
| | | APNSQYGTPSSFRTSPSSQFGSSSAHSSSLSKFRSVPSSPYGTLSAIRSTH |
| | | SSQYGTPSSFSDSTSSSHNGLPSHYPGSGFSGSSVNDQKSYTGNVFGQSHS |
| | | RVANGDQHARSYTLAGGNEISEPAKYDFNYDVSDGEQGVEFGQEESRDGEE |
| | | TNGSYHVLLPDGRRQRVQYTAGQYGYKPTISYENTGTLTTGRQQFSNGFYN |
| | | VQQSGSESQEHLGRSTGQNSYGGSNGYESGVGYQSGVGRRSRPAGSY |
| 31 | Solenopsis invicta | RSEPPINSYLPPRAGSSGANGGRTDLTTQYGAPDFNNGGGATSFSGNGAGD |
| | | GPSKLYDVPVRGNAGGNGLGRGNGFGGGQPSSSYGAPNGGSNENRGNGGRP |
| | | STSYGVPGANGNNGGGFGNGGDKGRPSTSYGVPDASGSSQGSFGNVGNGGR |
| | | PSTNYGVPGANGNGGGFGNAANEGKPSTSYGVPGANGNSQGGFGNGGRPST |
| | | GYGVPGANGNNGGGFGGRPSTSYGAPGANGNHRGGNGGNASPSTNYGVPGG |
| | | NNGNTNGKGRFNGGNSGGGPSNNYGVPNENAFGGGLSTSYGPPSRGGNGNS |
| | | GYPSGGSNGGSFVNNGANGYPSGGPNGNAGNFGDGRGGKGGGSSGEGYNDN |
| | | AQEGSTEPAKYEFSYKVKDQQTGSEYSHTETRDGDRAQGEFNVLLPDGRKQ |
| | | IVEYEADQDGFKPQIRYEGEANAGGGYSSGGSNDNNDGYSSGRPGSEAGGF |
| | | ANNSGFNGSGTNGGRSSGGPGDGNPGGFNSGGGGGYQSGRPAGQSFGRDND |
| | | GGLSGDIGGYFANSPSNNIGGSDSANVGSNRQNGGNGGYQY |
| 32 | Culex | KREAPLPGGSYLPPSNGGGAGGYPAAGPPSGSYGPPSNGNGNGNGAGGYPS |
| | quinquefasciatus | APSQQYGAPAGGAPSQQYGAPSNGNGGAGGYPSAPSQQYGAPNGNGNGGFG |
| | | GRPQAPSQQYGAPSNGNGGARPSQQYGAPNGGNGNGRPQTPSSQYGAPSGG |
| | | APSSQYGAPSGGAPSQQYGAPNGGNGNGRPQTPSSQYGAPSGGAPSQQYGA |
| | | PNGGNGNGRPQTPSSQYGAPSGGAPSSQYGAPSGGAPSSQYGAPAGGAPSS |
| | | QYGAPAGGAPSSQYGAPAGGAPSSQYGAPAGGAPSSQYGAPAGGAPSSQYG |
| | | APSSQYGAPAGGAPSSQYGAPAGGAPSSQYGAPSGGAPSSQYGAPSGGAPS |
| | | SQYGAPAGGAPSSQYGAPSGGAPSS |
| 33 | Bactrocera | RPEPPVNSYLPPSANGNGNGGGRPSSQYGAPGLGSNSNGNGNGNGGGRPSS |
| | cucurbitae | QYGVPGLGGNGNGNGNGGGGGRPSSSYGAPGLGGNGNGNGNGGGRPSSQYG |
| | | VPGLGGNGNGNGNGNGGGRPSSTYGAPGLRGNGNGNGNGNGRPSSTYGAPG |
| | | LGGNGNGNGNGNGRPSSTYGAPGLGGNGNGNGNGNGRPSSTYGAPGLNGNG |
| | | LGGGQKPSDSYGPPASGNGNGYSNGGNGNGNGGGRPGQEYLPPGRNGNGNG |
| | | NGGRGNGNGGGANGYDYSQGGSDSGESGIVDYEADQGGYRPQIRYEGEANN |
| | | GAGGLGGGAGGANGYDYEQNGNGLGGGNGYSNGQDLGSNGYSSGRPNGNGN |
| | | GNGNGNGNGYSGRNGKGRNGNGGGQGLGRNGYSDGRPSGQDLGDNGYASGR |
| | | PGGNGNGNGGNGNGYSNGNGYSNGNGNGTGNGGGQYNGNGNGYSDGRPGGQ |
| | | DNLDGQGYSSGRPNGFGPGGQNGDNDGNGYRY |
| 34 | Trichogramma | RPEPPVNSYLPPGQGGQGGFGGSGGRPGGGSPSNQYGPPNFQNGGGQNGGS |
| | pretiosum | GFGGNGNGNSFGPPSNSYGPPEFGSPGAGSFGGGRPQDTYGPPSNGNGNGN |
| | | GFGGNGNGGGRPSSRPSDSYGPPSSGNGFGGGNSGRPSESYGPPQNGGGSG |
| | | NGNQGGGNGFGNGGGRGGQGKPSDSYGPPNSGNRPGSSNGGGQQQNGFGGG |
| | | NGGRPSNTYGPPGGGNGGGRPGGSSGGFGGQNGGRPSDSYGPPSNGNGNGG |
| | | RPSNNYGPPNSGGGNGNGFGGSNGKPSNSYGPPSNGNGGGFGGSNGRPSNS |
| | | YGPPSGGNGGGFGGSSAVGRPGNSGSPSSSGSGFGGNGGASRPSSSYGPPS |
| | | NGGGFGNGGGSNGRPSSSYGPPNSGSNGGGFGGQNGNGRQNGNNGQGGFGG |
| | | QPSSSYGPPSNGNGFGGGGGSNGYPQNSQGGNGNGFGQGSGGRPSSSYGPP |
| | | SNGGGGGDNGYSSGGPGGFGGQPQDSYGPPPSGAVDGNNGFSSGGSSGDNN |
| | | GYSSGGPGGNGFEDGNDEPAKYEFSYEVKDEQSGSSFGHTEMRDGDRAQGE |
| | | FNVLLPDGRKQIVEYEADQDGFKPQIRYEGEANTGGAGGYPSGGPGGQGGN |
| | | GNGGYPSGGPSNGGFGGQNGGGNGGYPSGGPSGGGFGGQNGGSGGYPSGGP |
| | | SGGGFGGQGGFGGQNSGGNGGYSSGGPASGGFGGQNGGNGGYPSGGPSGGG |
| | | FGGQGGFGGQNSGGNGGYPSGGPSSGGFGGQNGGGGGNYPAGSGGDAEANG |
| | | GYQYS |
| 35 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 36 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 37 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 38 | Drosophila | QSGAGGRPSDTYGAPGGGNGGRPSDSYGAPGQGQGQGQGQGGYGGKPSDSY |
| | sechellia | GAPGGGNGNGGRPSSSYGAPGGGNGGRPSDTYGAPGGGNGGRPSDTYGAPG |
| | | GGGNGNGGRPSSSYGAPGQGQGNGNGGRPSSSYGAPGGGNGGRPSDTYGAP |
| | | GGGNGGRPSDTYGAPGGGNNGGRPSSSYGAPGGGNGGRPSDTYGAPGGGNG |
| | | NGSGGRPSSSYGAPAQGQGGFGGRPSDSYGAPGQNQKPSDSYGAPGSGNGS |
| | | AGRPSSSYGAPGSGPGGRPSDSYGP |
| 39 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 40 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 41 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 42 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 43 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 44 | Drosophila | YSSGRPGNGNGNGNGGYSSGRPGGQDLGPSGYSGGRPGGQDLGAGGYSNVK |
| | sechellia | PGGQDLGPGGYSGGRPGGQDLGRDGYSGGRPGGQDLGAGAYSNGRPGGNGN |
| | | GGSDGGRVIIGGRVIGGQDGGDQGYSGGRPGGQDLGRDGYSSGRPGGRPGG |
| | | NGQDSQDGQ |
| 45 | 3X FLAG | GDYKDDDDKDYKDDDDKDYKDDDDK |
| 46 | Alpha mating | MRFPSIFTAVLFAASSALAAPVNTTTEDETAQIPAEAVIGYSDLEGDFDVA |
| | factor precursor | VLPFSNSTNNGLLFINTTIASIAAKEEGVSLEKR |
| | protein sequence | |
| 47 | EA repeat | EAEA |
| 48 | Linker | SG |

1. A method for producing a composition comprising a recombinant resilin protein, comprising:
culturing a population of recombinant host cells in a fermentation, wherein said recombinant host cells comprise a vector comprising a secreted resilin coding sequence selected from the group consisting of SEQ ID NOS: 13-44, and wherein said recombinant host cells secrete a recombinant resilin protein encoded by said secreted resilin coding sequence; and
purifying said recombinant resilin protein from said fermentation.

2. The method of claim 1, wherein said recombinant resilin protein is a full-length or truncated native resilin.

3. (canceled)

4. (canceled)

5. (canceled)

6. The method of claim 1, wherein the recombinant resilin protein comprises an alpha mating factor secretion signal.

7. The method of claim 1, wherein the recombinant resilin protein comprises a FLAG-tag.

8. The method of claim 1, wherein the vector comprises more than one secreted resilin coding sequence.

9. The method of claim 1, wherein the recombinant host cells are yeast cells.

10. The method of claim 9, wherein the yeast cells are methylotrophic yeast cells.

11. (canceled)

12. The method of claim 1, wherein the recombinant host cells produce the recombinant resilin at a rate of greater than 2 mg resilin/g dry cell weight/hour.

13. The method of claim 1, wherein the recombinant host cells produce a secreted fraction of the recombinant resilin that is greater than 50% as compared to the total recombinant resilin protein expressed by the recombinant host cells.

14. The method of claim 1, wherein the recombinant host cells secrete the recombinant resilin at a rate of greater than 2 mg resilin/g dry cell weight/hour.

15. The method of claim 1, wherein greater than 80% of the recombinant resilin is outside of the recombinant host cells in said fermentation.

16. The method of claim 1, wherein the fermentation comprises at least 2 g recombinant resilin/L.

17. The method of claim 1, wherein purifying said recombinant resilin protein comprises generating a first pellet fraction and a first supernatant fraction by centrifuging the fermentation; and
isolating the recombinant resilin protein from the first pellet fraction.

18. The method of claim 17, wherein purifying said recombinant resilin protein further comprises:
adding a chaotrope to the first pellet fraction to generate a solution in which the recombinant resilin protein is soluble;
generating a second supernatant fraction and a second pellet fraction by centrifuging the first pellet fraction comprising said chaotrope; and
isolating the soluble full-length resilin from the second supernatant fraction.

19. A vector comprising a secreted resilin coding sequence.

20. (canceled)

21. (canceled)

22. (canceled)

23. (canceled)

24. (canceled)

25. (canceled)

26. The vector of claim 19, wherein the secreted resilin coding sequence encodes a recombinant resilin comprising one or more A-repeats or quasi-A-repeats.

27. The vector of claim 19, wherein the secreted resilin coding sequence encodes a recombinant resilin comprising one or more B-repeats or quasi-B-repeats.

28. The vector of claim 19, wherein the secreted resilin coding sequence encodes a recombinant resilin comprising either one or more A-repeats or quasi-A-repeats or one or more B-repeats or quasi-B repeats but not both.

29. The vector of claim 19, wherein the secreted resilin coding sequence encodes a recombinant resilin comprising one or more A-repeats or quasi-A-repeats and one or more B-repeats or quasi-B-repeats.

30-68. (canceled)

69. A composition comprising a gel comprising a recombinant resilin selected from the group consisting of SEQ ID NOs: 13-44.

70. The composition of claim 69, wherein the gel comprises full-length resilin.

71. The composition of claim 69, wherein the gel comprises a plurality of cross-linked recombinant resilins.
